SEQUENCE LISTING (14 sequences)

SEQ ID NO: 1 - protein, 480 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKSGLRSSTESMSTESMIRDVELAEEALPKKMGGLQNSRRCLCLSLFSFLLVAGATTLFCLLNFGVIGPNKEEKFPNGLPLISSMAQTLTLRSSSQNSSDKPVAHVVANHQAEEQLEWLSQRANALLANGMDLKDNQLVVPADGLYLIYSQVLFKGQGCPDYVLLTHTVSRFAISYQEKVSLLSAIKSPCPKDTPEGAELKPWYEPMYLGGVFQLEKGDLLSAEVNLPKYLDITESGQVYFGVIAL

SEQ ID NO: 2 - DNA, 45 bp, synthetic construct
  source 1..45: mol_type = other DNA
tccggactcagatctagcacagaaagcatgagcacggaaagcatg

SEQ ID NO: 3 - DNA, 39 bp, synthetic construct
  source 1..39: mol_type = other DNA
ggagggagaggggcgggatcctcacagagcaatgactcc

SEQ ID NO: 4 - DNA, 46 bp, synthetic construct
  source 1..46: mol_type = other DNA
ggatctatttccggtgaattcgccaccatggtgagcaagggcgagg

SEQ ID NO: 5 - DNA, 45 bp, synthetic construct
  source 1..45: mol_type = other DNA
ctgtgctagatctgagtccggacttgtacagctcgtccatgccgc

SEQ ID NO: 6 - DNA, 28 bp, synthetic construct
  source 1..28: mol_type = other DNA
ggaattcgccaccatgtctacaggggtg

SEQ ID NO: 7 - DNA, 24 bp, synthetic construct
  source 1..24: mol_type = other DNA
cgggatccagagacacaccacaca

SEQ ID NO: 8 - DNA, 37 bp, synthetic construct
  source 1..37: mol_type = other DNA
aaaacgacggccagtgaattcctggcttgagcaatcc

SEQ ID NO: 9 - DNA, 37 bp, synthetic construct
  source 1..37: mol_type = other DNA
gaaataacctagatcggatccgcactggaccctgaag

SEQ ID NO: 10 - DNA, 37 bp, synthetic construct
  source 1..37: mol_type = other DNA
gattacgccaagcttgcatgtgcttcagactttggtc

SEQ ID NO: 11 - DNA, 19 bp, synthetic construct
  source 1..19: mol_type = other DNA
aaagaggtggagggtgaag

SEQ ID NO: 12 - DNA, 19 bp, synthetic construct
  source 1..19: mol_type = other DNA
gcaggttcatgtcgcaaag

SEQ ID NO: 13 - DNA, 19 bp, synthetic construct
  source 1..19: mol_type = other DNA
acaccctacaagccagaac

SEQ ID NO: 14 - DNA, 19 bp, synthetic construct
  source 1..19: mol_type = other DNA
tcctaacatcagcagaccc

(19) United States
(12) Patent Application Publication
(10) Pub. No.: US 2025/0073235 A1
(43) Pub. Date: Mar. 6, 2025

(54) INHIBITION OF MAST CELL TUMOR NECROSIS FACTOR FOR THERAPEUTIC USE

(71) Applicants: Hao Xu, Purvis, MS (US); Tolulope Eunice Ayo, Safford, AZ (US)

(72) Inventors: Hao Xu, Purvis, MS (US); Tolulope Eunice Ayo, Safford, AZ (US)

(73) Assignee: The University of Southern Mississippi, Hattiesburg, MS (US)

(21) Appl. No.: 18/820,911

(22) Filed: Aug. 30, 2024

Related U.S. Application Data
(60) Provisional application No. 63/535,762, filed on Aug. 31, 2023.

(51) Int. Cl. A61K 31/506; A61K 31/145; A61K 45/06; A61P 37/08

(52) U.S. Cl. CPC A61K 31/506; A61K 31/145; A61K 45/06; A61P 37/08

(57) ABSTRACT

A method for inhibiting the release of tumor necrosis factor (TNF) from mast cells including administering an effective amount of an inhibitor to a subject; wherein the inhibitor is an inhibitor that selectively targets conventional mitogen activated protein kinase signaling pathways; and allowing the inhibitor to interact with mast cells or their signaling pathways involved in TNF release, thereby inhibiting TNF release. Also disclosed is a method for preventing the conversion of membrane-bound tumor necrosis factor (mTNF) to soluble tumor necrosis factor (sTNF) in mast cells using an inhibitor including administering an effective amount of the inhibitor to a subject; wherein the inhibitor is a chemical compound that selectively targets conventional mitogen activated protein kinase signaling pathways; and allowing the inhibitor to interact with mast cells or the enzymatic machinery responsible for the cleavage of mTNF within mast cells, thereby inhibiting the conversion of mTNF to sTNF.

### CROSS REFERENCE TO RELATED APPLICATIONS

This application claims the benefit of U.S. Provisional Application No. 63/535,762, filed on Aug. 31, 2023, the entire disclosure of which is hereby incorporated by reference as if set forth fully herein.

### STATEMENT OF GOVERNMENT INTEREST

This invention was made with government support under grant nos. 1R15AI133430-01 and 1R03AI169047-01 awarded by the National Institutes of Health. The government has certain rights in the invention.

### INCORPORATION OF MATERIAL OF XML SEQUENCE LISTING BY REFERENCE

The sequence listing submitted herewith as an XML file named “USM1018USSequenceListing” created on Aug. 27, 2024, which is 14 kilobytes in size, is hereby incorporated by reference in its entirety.

### BACKGROUND OF THE INVENTION

Mast cells have-emerged, besides monocytes/macrophages, NK cells, and T cells, as another key producer of tumor necrosis factor (TNF; also known formerly as TNFa and cachectin) [53-56], with profound physiological and pathological consequences. Mast cell-derived TNF was shown to mediate host defense against viral (e.g., herpes simplex virus) [57] and bacterial infections [58-60]. Mast cell-derived TNF was also found to promote the proliferation and activation of T cells [61, 62].

On the other hand, TNF in excess leads to a variety of inflammatory disorders [63]. For instance, reconstitution of mast cell-deficient mice indicates that mast cell-derived TNF causes allergic airway diseases (e.g., asthma) [64]. Also, persistent elevation of TNF is associated with severe asthma in 15-30 million individuals (worldwide) that do not respond to the typical treatment of corticosteroid [65, 66]. To help this difficult-to-treat patient group, it is critical to fully understand the intricate regulation of TNF expression and exocytosis that is unique to mast cells.

Studies of model systems such as RBL-2H3 and BMMC (bone marrow-derived mast cells) have already yielded a great wealth of information regarding the life cycle of TNF in mast cells. In contrast to macrophages that do not express any basal level of TNF [67-69], mast cells pre-store TNF in secretory granules that can be rapidly released upon IgE receptor/FcFRI-dependent activation, in a process called degranulation [70]. This is initiated by FcRI crosslinking, which triggers phospholipase Cγ-dependent production of secondary messenger inositol 1,4,5-trisphosphate, leading to a sharp rise of intracellular Ca²⁺ [71], a key signal for regulated fusion of secretory granules in mast cells. In parallel, FcεRI crosslinking boosts the transcription of the TNF gene [54,72-78], exploiting NFAT (nuclear factor of activated T-cells)-dependent assembly of enhancesomes at the proximal promoter region [79, 80]. Newly synthesized TNF then undergoes constitutive secretion via Golgi-derived vesicles or recycling endosomes.

Like any exocytic event in eukaryotic cells, mast cell exocytosis (degranulation or constitutive secretion) requires membrane-anchored SNAREs (soluble N-ethylmaleimide sensitive factor attachment protein receptors) from apposing membranes (e.g., granule/vesicle vs the plasma membrane) to assemble into a 4-helical bundle (aka the trans-SNARE complex) [81]. The complete assembly of the exocytic trans-SNARE complex, which is coordinated by Munc13 proteins and catalyzed by members of the Munc18 family, causes the mixture of lipid bilayers and subsequently content release. Knocking out Munc13-4 from RBL-2H3 mast cells resulted in a severe defect in exocytosis that was accompanied by reduced TNF level [82], demonstrating a probable correlation between exocytosis and TNF synthesis.

While the importance of TNF in human health and disease has been widely acknowledged, most researchers in the field believe soluble TNF (sTNF) gets released to perform a long-distance function whereas the full-length, membrane-bound TNF (mTNF) is anchored onto the cell surface and can only have limited local impact (FIG. 1A). In addition, sTNF and mTNF have exhibited overlapping but distinct biochemical/signaling properties. Both mTNF and sTNF are known to bind TNF receptor 1 (TNFR1) and TNFR2, yet mTNF was found to be the prime activator of TNFR2 [25, 26]. On the other hand, there is evidence that sTNF but not mTNF is responsible for TNF autoregulation in RBL-2H3 cells (a tumor analog of mucosal mast cells) [27]. An additional difference between the two forms of TNF is that mTNF emanated from the surface of a cell could act as a receptor that transduces a “reverse signal” back into the host cell (when bound to TNFR or anti-TNF) [28-30].

The differences in their signaling properties are also manifested at the physiological/pathological level. For example, expression of sTNF in cardiomyocytes was found to cause dilation of the left ventricle in mice, whereas mTNF resulted in a concentric hypertrophic cardiac phenotype [31, 32]. mTNF and sTNF have also demonstrated diametrically opposing effects on both tumor growth and myeloid content [33]. Therefore, distinguishing the regulation of mTNF expression/release vs sTNF expression/release will enable development of therapeutic strategies that can specifically target an mTNF-linked disease or an sTNF-linked disease.

### SUMMARY OF THE INVENTION

The present invention demonstrates that mTNF can be released from mast cells via exosomes inside MVBs (multivesicular bodies) (FIG. 2). This demonstrates that 1) mTNF derived from mast cells could have unexpected signaling roles in allergic diseases (e.g., asthma) and immunity. Also, there was evidence that mTNF was released from mast cells in response to an allergen (triggering allergic inflammation) while sTNF was released in response to pathogen (triggering innate or adaptive immunity). Finally, it was shown that different Munc13 proteins likely mediate the release of mTNF and sTNF, respectively (FIGS. 20A-20B) allowing differentiated targeting to control the release of mTNF vs sTNF.

This invention provides new therapeutic strategies that alleviate TNF associated diseases by developing new drug targets to inhibit and/or promote release of mTNF and sTNF. Without wishing to be bound by theory, the evidence suggests that mast cell-derived mTNF could be responsible for allergic inflammation whereas sTNF could be responsible for adaptive immunity.

This evidence leads to the provision of therapeutic methods to inhibit mTNF release (FIG. 1B, pathway A) while preserving sTNF release (FIG. 1B, pathway B), which would treat asthma without jeopardizing the patient's immunity.

Anti-TNF blockades currently used to treat TNF related diseases were not designed to distinguish sTNF from mTNF. This invention provides new therapeutic strategies for disrupting distinct secondary pathways and properties for TNF.

In another aspect, the present invention relates to a novel RBL-2H3 cell line that was developed to stably express mCherry-TNF. This particular design—tagging a fluorescent protein or luminescent protein at the N-terminus of TNF—permits straightforward quantification of the secretion of (exosomal) mTNF and can be utilized or adapted for high throughput screens for drugs that inhibit mTNF release from mast cells and possibly other TNF producing cells including but not limited to neutrophils and macrophages.

The present invention may be described by the following sentences:

1. In a first aspect, the present invention relates to a method for inhibiting the release of tumor necrosis factor (TNF) from mast cells, comprising steps of.
a) administering an effective amount of an inhibitor to a subject; wherein the inhibitor is an inhibitor that selectively targets conventional mitogen activated protein kinase signaling pathways; and
b) allowing the inhibitor to interact with mast cells or their signaling pathways involved in TNF release, thereby inhibiting TNF release.

2. The method of sentence 1, wherein the mitogen activated protein kinase inhibitor may be selected from the group consisting of MEK inhibitors, BRAF inhibitors, dual BRAF and MEK inhibitors, ERK Inhibitors, JNK Inhibitors, p38 MAPK Inhibitors, and TNF-alpha converting enzyme (TACE) inhibitor.

3. The method of sentence 1, wherein the inhibitor may be selected from the group consisting of INK inhibitors.

4. In a second aspect, the present invention may relate to a method for preventing the conversion of membrane-bound tumor necrosis factor (mTNF) to soluble tumor necrosis factor (sTNF) in mast cells using an inhibitor, comprising steps of:
a) administering an effective amount of the inhibitor to a subject; wherein the inhibitor is a chemical compound that selectively targets conventional mitogen activated protein kinase signaling pathways; and
b) allowing the inhibitor to interact with mast cells or the enzymatic machinery responsible for the cleavage of mTNF within mast cells, thereby inhibiting the conversion of mTNF to sTNF.

5. The method of sentence 4, wherein the mitogen activated protein kinase inhibitor may be selected from the group consisting of MEK inhibitors, BRAF inhibitors, dual BRAF and MEK inhibitors, ERK Inhibitors, JNK Inhibitors, p38 MAPK Inhibitors, and TNF-alpha converting enzyme (TACE) inhibitor.

6. The method of sentence 4, wherein the inhibitor is selected from the group consisting of JNK inhibitors.

7. In a third aspect, the present invention relates to a method for treating a disease or disorder caused by mast cell tumor necrosis factor, comprising a step of:
administering an effective amount of a pharmaceutical composition to a subject wherein the pharmaceutical composition comprises an inhibitor that selectively targets conventional mitogen activated protein kinase signaling pathways.

8. The method of sentence 7, wherein the disease may be selected from the group consisting of asthma, allergic rhinitis, atopic dermatitis, inflammatory bowel disease, rheumatoid arthritis, psoriasis, anaphylaxis, systemic mastocytosis, chronic urticaria, and autoimmune disorders.

9. The method of sentence 7, wherein the mitogen activated protein kinase inhibitor may be selected from the group consisting of MEK inhibitors, BRAF inhibitors, dual BRAF and MEK inhibitors, ERK Inhibitors, JNK Inhibitors, p38 MAPK Inhibitors, and TNF-alpha converting enzyme (TACE) inhibitor.

10. The method of sentence 7, wherein the inhibitor may be selected from the group consisting of TNF-alpha converting enzyme inhibitor KP-457; ERK1/2 extracellular signal-regulated kinase1/2 Ravoxertinib (GDC-0994); and small molecule inhibitor JNK-IN8.

11. The method of sentence 7, wherein the inhibitor may be selected from the group consisting of INK inhibitors.

12. In a third aspect, the present invention relates to a method of producing RBL-2H3 cells that stably express mCherry-TNF comprising steps of:
amplifying rat TNF using 5-TCCGGACTCAGATCTAGCACAGAAAGCATGAGCAC GGAAAGCATG-3′ and 5′-GGAGGGAGAGGGGCGGGATCCTCACAGAGCAATGACTCC-3′ as forward and reverse primers,
amplifying mCherry from pCDH-TNF-SBP-mCherry using 5′-GGATCTATTTCCGGTGAATTCGCCACCATGGTGAGCAAGGGCGAGG-3′ and 5′-CTGTGCTAGATCTGAGTCCGGACTTGTACAGCTCGTCCATGCCGC-3′ as forward and reverse primers,
joining the mCherry and rat TNF amplicons together by SOEing PCR;
gel purifying; digesting pLVX-IB-EmGFP with EcoRI-HF and BamHI-HF to obtain a linearized pLVX-IB vector;
recombining the linearized pLVX-IB vector with the Mcherry-TNF using cold fusion cloning to obtain pLVX-IB-mCherry-TNF; and
transfecting HEK293 FT cells with the pLVX-IB-mCherry-TNF to generate RBL-2H3 cells that stably express mCherry-TNF.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Shows TNF secretion from mast cells. (A) Domain structure of TNF. (A) Full-length TNF has a cytoplasmic domain (CD) and transmembrane domain (TD) which is removed at the TACE (tumor necrosis factor activating enzyme) cleavage site when TACE is present. This releases soluble TNF. (B) Mast cells activated by allergens (via IgE receptor/FceRI) rapidly secrete prestored mediators from cytoplasmic granules. Allergens or pathogens [via toll-like receptors (TLRs)] could also trigger the production of TNF that is subsequently released via constitutive secretory carriers.

FIG. 2. Shows measured mTNF exocytosis from MVBs. Mast cells transfected with a mCherry-TNF construct transiently express the chimeric protein with the mCherry tag facing either the cytosol, or the interior of the exosomes inside MVBs (A). Exocytosis of MVB would lead to mCherry signal accumulation in the supernatant whereas exocytosis of secretory vesicles would not. TACE cleaves and releases TNF molecules that have reached the plasma membrane (PM). In (B), RBL-2H3 cells stably expressing mCherry-TNF were sensitized with 0.5 mg/ml anti-TNP IgE (lane 2) or control buffer (lane 1) for 3 hours before addition of 25 ng/ml TNF-βSA. At 30 min., fluorescent signals in the supernatant and cell pellet were measured. The % of mCherry-TNF secretion was averaged from 6 biological repeats. Error bars=std. ****p<0.0001. In (C), RBL-2H3 mast cells co-transfected with CD63-GFP (MVB marker) and TNF-mCherry were examined under a confocal microscope. Scale bar=5 um.

FIG. 3. Effect of Vesicle-Associated Membrane Protein or Synaptobrevin (VAMP) knockdown in allergen-triggered mast cell exocytosis. (A) small interfering RNA (siRNA)-mediated knockdown of VAMP in RBL-2H3 cells was assessed via RT-qPCR. At least 5 biological repeats were conducted to calculate averages and standard deviations. (B-E) Secretion of each mast cell mediator from cells treated with VAMP-specific siRNA was normalized against the secretion of the same mediator from cells mock treated with non-target siRNA (lane 1). Averages and standard deviations were calculated from at least 4 biological repeats. **p<0.01; ***p<0.001

FIG. 4. Rescue of secretion defects of VAMP8KD cells. RBL-2H3 cells were treated with non-targeting (NT) siRNA (lane 1) or VAMP8 siRNA (V8; lanes 2 to 4). VAMP8KD cells were then separated into three aliquots, two of which were either transduced with lentiviruses carrying RNAi-resistant VAMP8Ala (T47A, T53A, S54A) (V8A; lane 4) or mock infected with lentiviruses without the transgene (lane 3). Cells were then stimulated by IgE/TNP-BSA (Bovine Serum Albumin). Normalized secretion from each condition was calculated from 7 independent experiments. Error bars represent standard deviations. **p<0.01; ***p<0.001; ****p<0.0001

FIG. 5. Spatial distribution of TNF and serotonin in RBL-2H3 cells. (A-D) RBL-2H3 cells stably expressing mCherry-TNF were transfected with pEGFP-VAMP8, fixed in 4% paraformaldehyde and immunostained with an antibody specific for serotonin (monoclonal; 1:100 dilution). Goat anti-mouse IgG (Alexa Fluor™ 568) was used as the secondary antibody. (E) Pearson's correlation coefficient averaged from 28 cells is presented, with error bars representing SEM. ***p<0.001. Scale bar, 20 μm.

FIG. 6. Analyzing VAMP3 knockouts in RBL-2H3 cells. (A) Cell lysates from control RBL-2H3 cells or VAMP3^{KO} clones were run on 15% sodium dodecyl sulfate-polyacrylamide gel electrophoresis (SDS-PAGE) and immunoblotted with anti-actin (1:100 dilution) and anti-VAMP3 (1:500 dilution) antibodies. Five out of the 9 clones are shown. (B and C) The absolute amount of TNF released from control and VAMP3^{KO} (Clone #16) cells 30 min. after FceRI-mediated activation was 31.4 (+/−16.6) pg. and 42.5 (+/−26.9) pg. respectively. The absolute amount of TNF released from control and VAMP3^{KO} cells 24 hours after FceRI-mediated activation was 22.9 (+/−8.5) pg. and 33.9 (+/−16.9) pg. respectively. To reduce noise caused by variation in cell density, cell number, and passage number between biological replications, and the time gap between data collection (e.g., the 30 min. secretion assays and the 24 hour secretion assays were conducted months apart), secretion of TNF from control or VAMP3KO cells 30 min. (B) or 24 hours (C) after activation was expressed as the percentage of the ELISA signal released in the supernatant relative to the total signal. Averages and standard deviations from at least 4 independent experiments are presented. (D) Expression of exocytic v-SNAREs in VAMP3^{KO} cells. Cell lysates from control RBL-2H3 cells or VAMP3^{KO} cells were subject to SDS-PAGE and subsequent immunoblotting using VAMP specific antibodies.

FIG. 7. Munc18a and Munc18b in the control of exocytosis in RBL-2H3 cells. Allergen-induced exocytosis of β-hexosaminidase, serotonin, histamine and TNF in stable Munc18a^{KD} cells (A) and Munc18b^{KD} cells (B) was measured, and the values were normalized against that of the control cells (blue) that were stimulated in the same manner. Averages and standard deviations from at least 5 biological repeats are presented. *p<0.05; **p<0.01; ***p<0.001 FIG. 8. RNAi-resistant Munc18b restored allergen-triggered exocytosis in Munc18KD RBL-2H3 cells. Munc18b^{KD} cells were separated into three aliquots, two of which were either transduced with lentiviruses carrying RNAi-resistant Munc18b (lane 4) or mock infected with lentiviruses without the transgene (lane 3). These cells were then stimulated by IgE/TNP-BSA, and the level of their secretion was normalized against that of a control RBL-2H3 cell line (lane 1) that was stimulated in the same manner. Averages and standard deviations from at least 5 independent experiments are presented. *p<0.05; **p<0.01; ***p<0.001

FIG. 9. Spatial distribution of Munc18a and Munc18b in RBL-2H3 cells. RBL-2H3 cells stably expressing mCherry-TNF were fixed in 4% paraformaldehyde and immunostained with (5 mg/ml) mouse anti-Munc18a antibodies (A) or (3 mg/ml) rabbit anti-Munc18b antibodies (B). Goat anti-mouse IgG (Alexa Fluor™ 488) and goat anti-rabbit IgG (Alexa Fluor™ 405) were used as secondary antibodies. The colocalization of blue Munc18b signal and red TNF signal (C) gave rise to a purple signal in (D). (E) Pearson's correlation coefficient averaged from 46 cells is presented, with error bars representing SEM. “ns” means p>0.05; ****p<0.0001. Scale bar, 20 μm.

FIG. 10. Quantification of VAMP protein levels in resting RBL-2H3 cells.

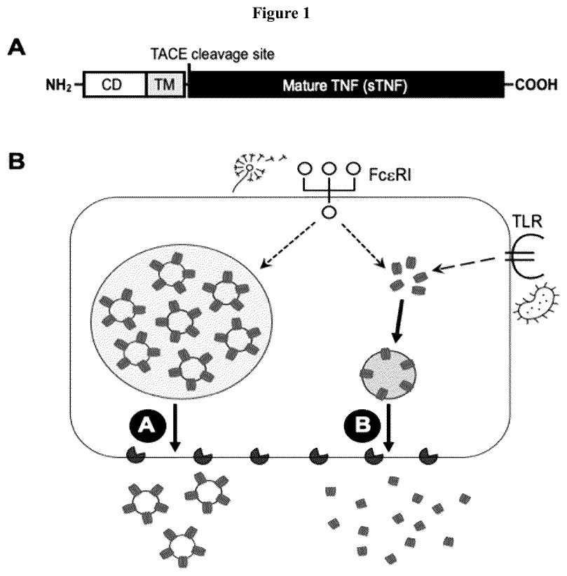

(A) Recombinant MBP-TEV-VAMP proteins (10 ng and 50 ng) were subject to 15% SDS-PAGE and immunoblotting with antibodies specific for VAMP2 (V2), VAMP3 (V3), VAMP7 (V7) or VAMP8 (V8). (B) MBP-TEV-VAMP were treated with TEV (to remove MBP) and increasing amounts of TEV-treated samples (from 0.5 ng to 6 ng) were subject to SDS-PAGE and immunoblotting side-by-side with RBL-2H3 cell lysates [a total protein of 10 μg (for V8), 50 μg (for V2 and V3), or 150 μg (for V7) was used]. The amount of VAMP proteins in RBL-2H3 lysates was determined via densitometry using BioRad Image Lab 6.0™ software. (C) Molar concentrations of each VAMP protein in RBL-2H3 cell lysates were used to calculate the relative expression of these VAMP proteins in resting RBL-2H3 cells. Averages from 3 biological replicates are presented.

FIG. 11. Allergen-triggered mast cell exocytosis. Control RBL-2H3 cells as specified were sensitized with anti-TNP IgE for 3 hours and then activated by TNP-BSA for 1 hour. The percentage of total β-hexosaminidase (A), serotonin (B), histamine (C), or TNF (D) that was released into the supernatant was measured. Averages and standard deviations were calculated from at least 5 biological replicates.

FIG. 12. Rescue of VAMP3KO phenotype. Control RBL-2H3 cells (Ctrl) and VAMP3^{KO} cells were either transduced with lentiviruses carrying Emerald Green Fluorescent Protein (EmGFP) (mock) or VAMP3-EmGFP. (A) These cells were challenged by IgE/TNP-BSA, with 0-hexosaminidase secretion measured at the 30 min time point. Averages and standard deviations from 5 biological repeats are presented. (B) Lysates harvested from resting cells were subjected to SDS-PAGE and immunoblotting anti-actin (1:100 dilution) and anti-VAMP3 (1:500 dilution). ***p<0.001.

FIG. 13. Rescue of Munc18a^{KD} phenotype. Munc18a^{KD} cells were separated into three aliquots, two of which were either transduced with lentiviruses carrying RNAi-resistant Munc18a (lane 4) or mock infected with lentiviruses without the transgene (lane 3). These cells were then stimulated by IgE/TNP-BSA, and the value of their secretion was normalized against that of a control RBL-2H3 cell line (lane 1) that was stimulated the same way. Averages and standard deviations from at least 5 independent experiments are presented. ***p<0.001; ****p<0.0001

FIG. 14. Spatial distribution of TNF and VAMP8 in RBL-2H3 cells. RBL-2H3 cells stably expressing mCherry-TNF were fixed in 4% paraformaldehyde and immunostained with (A-C) monoclonal anti-VAMP8 (1:50 dilution; SantaCruz #166820) or (D-F) polyclonal anti-VAMP8 (4 μg/ml). Goat anti-mouse IgG (Alexa Fluor™ 488; Invitrogen #A11001) or donkey anti-rabbit IgG (Alexa Fluor™ 405) were used as the secondary antibodies (1:200 dilution). (G) Pearson's correlation coefficient averaged from 17 cells is presented, with error bars representing SEM. ****p<0.0001. Scale bar, 20 μm.

FIG. 15. TNF exocytosis correlates with TNF level. (A) TNP-BSA/IgE (anti-TNP)-triggered TNF exocytosis from Munc13-4^{KO} cells, stable Munc18a^{KD} cells, stable Munc18b^{KD} cells, or VAMP8KD cells (via siRNA) was measured 1 hour after stimulation, and the values were normalized against those of their respective control cells that were treated in the same manner. (B) Likewise, total TNF protein levels were calculated for activated KO or KD cells and normalized against those of the control cells. Averages and standard deviations from at least 4 biological repeats are presented. *p<0.05; **p<0.01; ***p<0.001.

FIG. 16. TNFR1 is expressed and upregulated in activated RBL-2H3 cells. RBL-2H3 cells sensitized with anti-TNP IgE or buffer were incubated with TNP-BSA for 1 hour. (A) Mast cell degranulation was measured and expressed as the percentage of 0-hexosaminidase in the supernatant. Meanwhile, lysates from activated (with IgE) or mock treated (with buffer) cells were subjected to reverse transcription followed by either qPCR to quantify the relative amount of TNFR mRNA (B) or regular PCR to visually compare the expression of TNFR1 and TNFR2 on 2% agarose gel (lanes 1 to 4). In lanes 5 and 6, one L of H₂O and rat universal cDNA were used respectively as the templates.

FIG. 17. TNF feedback loop is mediated by TNFR1. (A) RBL-2H3 cells sensitized with anti-TNP IgE were challenged with TNP-BSA along with specified amounts of sTNFR1 or buffer (PBS). At the 1 hour time point, total TNF protein levels in all three conditions were measured using an Enzyme-linked immunosorbent assay (ELISA). The total TNF in activated cells subtracted from that in resting cells was set at 1 (lane 1) and used to calculate the relative values of TNF production in the presence of sTNFR1 (lanes 2 and 3). Averages and standard deviations from 3 biological repeats are presented. **p<0.01; ***p<0.001. (B) Model of TNF autocrine loop. Mast cells elicited by allergens undergo calcium dependent exocytosis of full length, membrane-bound TNF (depicted in blue) that is prestored in secretory apparatuses such as MVB (1). Allergens also boost de novo synthesis of TNF, which is deposited into the endoplasmic reticulum (ER) (omitted for simplicity) and subsequently delivered onto the plasma membrane via the classical secretory pathway (2). Upon TACE/Adam17 (depicted in red)-dependent cleavage, soluble TNF is released to the extracellular space, where it binds TNFR1 to promote additional TNF production. TNF proteins that have escaped proteolysis can be internalized and delivered to MVB for storage (3).

FIG. 18. TACE inhibitor KP-457 reduces TNF proliferation. RBL-2H3 cells sensitized with anti-TNP IgE were challenged with TNP-BSA along with specified amounts of KP-457 or solvent (DMSO). At the 1 hour time point, 0-hexosaminidase secretion (A), released TNF (recovered from the supernatant) (B) and total TNF protein level (C) were measured, and the values were normalized against that of the solvent control (lane 2). Averages and standard deviations from at least 5 biological repeats are presented. *p<0.05; **p<0.01; ***p<0.001; ****p<0.0001.

FIG. 19. Effect of Mitogen-Activated Protein Kinase (MAPK) inhibitors on exocytosis and TNF production. Where indicated, MAP kinase inhibitors including (A) Doramapimod/BIRB 796 (IC50 of 38 nM for p38α, 65 nM for p38β, 200 nM for p38γ, and 520 nM for p38δ), (B) AX-15836 (IC50 of 8 nM for ERK5), (C) Ravoxertinib/GDC-0994 (IC50 of 6.1 nM and 3.1 nM for ERK1 and ERK2 respectively), and (D) JNK-IN-8 (IC50 of 4.7 nM, 18.7 nM, and 1 nM for JNK1, JNK2, and JNK3, respectively) were added to sensitized RBL-2H3 cells 30 min before activation by TNP-BSA. Total TNF, TNF secretion (% of total TNF in the supernatant), and β-hexosaminidase secretion (% of total β-hexosaminidase activity in the supernatant) were measured and normalized against that of the mock treatment (DMSO). Averages and standard deviations from 3 to 5 biological repeats are presented. *p<0.05; **p<0.01.

FIGS. 20A-20B Effect of Munc13 Proteins on the Release of mTNF and sTNF. These figures show that different Munc13 proteins likely mediate the release of mTNF and sTNF, respectively (FIGS. 20A-20B) allowing differentiated targeting to control the release of mTNF vs sTNF.

FIG. 21 is the amino acid sequence of mCherry-TNF (SEQ ID NO: 1).

### DETAILED DESCRIPTION OF THE INVENTION

In a first aspect, the present invention relates to methods for inhibiting the release of tumor necrosis factor (TNF) from mast cells, comprising steps of.
a) administering an effective amount of an inhibitor to a subject; wherein the inhibitor is an inhibitor that selectively targets conventional mitogen activated protein kinase signaling pathways; and
b) allowing the inhibitor to interact with mast cells or their signaling pathways involved in TNF release, thereby inhibiting TNF release.

In a second aspect, the present invention relates to methods for reducing or preventing the conversion of membrane-bound tumor necrosis factor (mTNF) to soluble tumor necrosis factor (sTNF) in mast cells using an inhibitor, comprising steps of:
a) administering an effective amount of the inhibitor to a subject; wherein the inhibitor is an inhibitor that selectively targets conventional mitogen activated protein kinase signaling pathways; and
b) allowing the inhibitor to interact with mast cells or the enzymatic machinery responsible for the cleavage of mTNF within mast cells, thereby inhibiting the conversion of mTNF to sTNF.

In a third aspect, the present invention relates to methods for treating a disease or disorder caused by mast cell tumor necrosis factor, comprising a step of:
administering an effective amount of a pharmaceutical composition to a subject wherein the pharmaceutical composition comprises an inhibitor that selectively targets conventional mitogen activated protein kinase signaling pathways.

Suitable examples of the inhibitors of the present invention that selectively target conventional nitrogen activated protein kinase signaling pathways may include MEK Inhibitors: Trametinib (Mekinist), Cobimetinib (Cotellic), Binimetinib (Mektovi), Selumetinib (AZD6244); BRAF Inhibitors: Vemurafenib (Zelboraf), Dabrafenib (Tafinlar), and Encorafenib (Braftovi); Dual BRAF and MEK Inhibitors: Dabrafenib+Trametinib (Tafinlar+Mekinist), Encorafenib+Binimetinib (Braftovi+Mektovi), Vemurafenib+Cobimetinib (Zelboraf+Cotellic); ERK Inhibitors: SCH772984, MK-8353 (LXH254), Ulixertinib (BVD-523, VRT752271), and Ravoxertinib (GDC-0994); JNK Inhibitors: SP600125, and JNK-IN-8; p38 MAPK Inhibitors: SB203580, BIRB 796, PH-797804, VX-745; and TNF-alpha Converting Enzyme (TACE) Inhibitor: KP-457.

The methods of the present invention may include treating a disease or disorder caused by mast cell tumor necrosis factor. Examples of diseases or disordered caused by mast cell tumor necrosis factor include:

Asthma: Mast cell-derived TNF can promote airway inflammation, bronchoconstriction, and mucus production, contributing to the development and exacerbation of asthma.

Allergic rhinitis: Mast cell TNF is involved in the inflammation of the nasal mucosa, leading to symptoms such as nasal congestion, sneezing, and itching in allergic rhinitis.

Atopic dermatitis: TNF released by mast cells can contribute to skin inflammation, barrier dysfunction, and itching in atopic dermatitis, a chronic inflammatory skin condition.

Inflammatory bowel disease (IBD): Mast cell TNF can participate in the inflammation and tissue damage seen in Crohn's disease and ulcerative colitis, two forms of IBD.

Rheumatoid arthritis: Mast cell-derived TNF plays a role in joint inflammation and destruction in rheumatoid arthritis, an autoimmune disease affecting the joints.

Psoriasis: Mast cell TNF contributes to the inflammation and abnormal skin cell proliferation characteristic of psoriasis, a chronic autoimmune skin condition.

Anaphylaxis: Mast cell-derived TNF can contribute to the severe systemic allergic reaction known as anaphylaxis, which can manifest with symptoms such as hives, respiratory distress, and low blood pressure.

Systemic mastocytosis: This rare disorder is characterized by the abnormal accumulation and activation of mast cells. Mast cell-derived TNF can contribute to the symptoms observed in systemic mastocytosis, including skin lesions, gastrointestinal disturbances, and anaphylactic episodes.

Chronic urticaria: Mast cell TNF is involved in the generation of hives and the chronic inflammation seen in chronic urticaria, a skin condition characterized by recurrent episodes of itching and wheals.

Autoimmune disorders: Mast cell-derived TNF can contribute to the pathogenesis of various autoimmune diseases, such as systemic lupus erythematosus, multiple sclerosis, and autoimmune thyroiditis.

Neurological pain: Mast cell-derived TNF has been linked to neuroinflammation and pain in fibromyalgia syndrome. See e.g. Theoharides, 2019 Frontier in Cellular Neuroscience and Gu, 2015, Journal of biological regulators & homeostatic agents.

The present invention also relates to methods for reducing mTNF without reducing overall TNF mRNA levels. There are two major steps in protein synthesis in a living organism: 1) transcription (mRNA production), and 2) translation (protein production using mRNA as a template). In allergic reactions, the binding of the allergens to the IgE receptor at the mast cell surface upregulates TNF production at the transcriptional level. However, the present application demonstrates that autoregulation of TNF production, mediated by sTNF and the TNF receptor at the cell surface, is mediated by inhibitors, such as JNK-IN-8 at the post transcriptional level.

In the present invention, the involvement of various fusion catalysts in TNF production was determined. A strong correlation between the total TNF level and TNF exocytosis in RBL-2H3 cells was observed. Quantitative reverse transcription polymerase chain reaction (RT-qPCR) showed that TNFR1 (TNF receptor 1) is the sole TNFR expressed in these cells, and that its transcription is upregulated upon allergen-mediated activation. Importantly, the addition of soluble TNFR1 inhibits antigen-elicited TNF production in a dosage-dependent fashion. Likewise, TNF production is diminished in the presence of TACE (TNFα Converting Enzyme) inhibitor KP-457, which prevents the generation of soluble TNF (sTNF). Together, these findings indicate that sTNF and TNFR1 function as autocrine agent and receptor respectively at the mast cell surface to boost TNF proliferation during allergic inflammation.

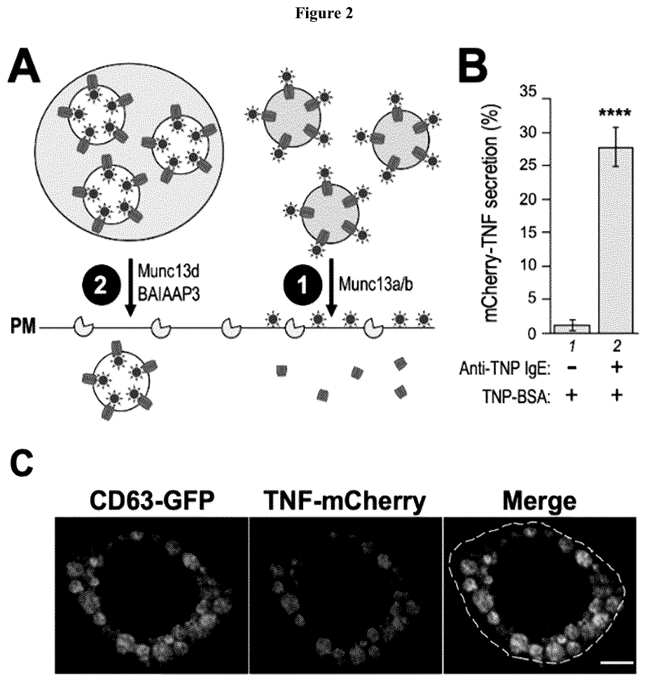

The assay employed by the present invention requires the generation of a fluorescently tagged TNF construct (mCherry or green fluorescent protein (GFP)), such that elease of the fluorescent signal is only possible when the full-length TNF is released from a multivesicular body (MVB) (FIG. 2A). FIG. 2B shows that full-length TNF could be easily released via the unconventional, multivesicular body (MVB)-mediated pathway. This assay provides a rapid measurement of mTNF (membrane-bound TNF) release from TNF producing cells under various conditions. It also serves as a convenient and new way to quantify the fusion between MVB and the plasma membrane, which leads to the release of exosomes (small vesicles inside MVB) that carry a variety of physiologically/pathologically important cargos including microRNA.

The core exocytic fusion machinery is composed of members of the SNARE family and the SM family. In close vicinity, SNAREs rooted in the plasma membrane/target membrane (t-SNAREs) and their cognate partner rooted in the vesicle/granule (v-SNARE) can form a 4-helical, fusogenic bundle (aka a trans-SNARE complex) [6-8]. SM proteins, which bind both the t- and v-SNAREs, accelerate the initiation and completion of trans-SNARE complex formation, and contribute to the protection of the already formed trans-SNARE complexes [9-12]. Each exocytic event is catalyzed by a unique set of SNARE-SM proteins [13]. In neurotransmission, for instance, the fusion of synaptic vesicles to the presynaptic membrane in the active zone is mediated by two t-SNAREs [i.e., SNAP-25 (contributing 2 helices) and syntaxin1 (contributing 1 helix)], one v-SNARE (i.e., VAMP 2), and one SM protein (i.e., Munc18a/Munc18-1) [14]. However, the SNARE-SM machinery underlying mast cell exocytosis has been more elusive, due in part to the expression of a multitude of exocytic SNAREs [e.g., SNAP-23, syntaxin3, 4, and VAMP2, 3, 7, 8] [15-23] and all three exocytic SM proteins (Munc18a, b, c) [24-27] in these cells. Reconstituted studies indicate that the SNAREs described above could form up to 8 functional trans-SNARE complexes, at least 4 of which act synergistically with Munc18 proteins [28]. These observations point to the existence of multiple, distinct exocytic events in mast cells.

Coincidentally, mast cell granules are strikingly heterogenous at the ultrastructural level [29] and exhibit different protein contents and accessibility to tracers [30]. It is conceivable that there are different populations of secretory carriers within a mast cell, each containing a different set of mediators. Consistent with this notion, the tricyclic antidepressant drug amitriptyline (Elavil) was found to specifically inhibit histamine release from activated mast cells, without any influence on serotonin release [31]. Of all the SNARE or SM proteins that have been identified, SNAP-23, Munc18a, and Munc18b are the only few that have been unequivocally shown to mediate allergen-dependent mast cell exocytosis [21, 26].

To delineate the distinct exocytic pathways in mast cells, allergen-triggered release of β-hexosaminidase, histamine, serotonin, and TNF from RBL-2H3 cells (a tumor analog of mucosal mast cells) was systematically monitored. All of them are prestored mediators except TNF, which is also rapidly synthesized upon mast cell activation [5, 32-35]. Using knockdown (KD) and knockout (KO) approaches, the expression of four exocytic v-SNAREs (e.g., VAMP2, 3, 7, 8) and two exocytic SM proteins (e.g., Munc18a, b) were inhibited one at a time, and any reversable phenotypes associated with the release of b-hexosaminidase, histamine, serotonin, or TNF were documented.

In another aspect, the present invention relates to a method for generating RBL-2H3 cells stably expressing mCherry-TNF. More specifically, to generate RBL-2H3 cells stably expressing mCherry-TNF, a TNF was amplified from TNF_Ora13562C_pMAL-c5E (GenScript) using using 5′-TCCGGACTCAGATCTAGCACAGAAAGCATGAGCACGGAAAGCATG-3′ (SEQ ID NO: 2) and 5′-GGAGGGAGAGGGGCGGGATCCTCACAGAGCAATGACTCC-3′ (SEQ ID NO: 3) as forward and reverse primers. mCherry was amplified from pCDH-TNF-SBP-mcherry (Addgene #65283, gift from Franck Perez) using
5′-GGATCTATTTCCGGTGAATTCGCCACCATGGTGAGCAAGGGCGAGG-3′ (SEQ ID NO: 4) and
5′-CTGTGCTAGATCTGAGTCCGGACTTGTACAGCTCGTCCATGCCGC-3′ (SEQ ID NO: 5) as forward and reverse primers.

Each PCR was conducted in a 50 μl mixture that contained 100 ng of the template DNA, 200 μM dNTPs, 5 μl of 10×pfu buffer, 5 units of Phusion DNA polymerase (G-Biosciences), and 200 nM of each primer. The mCherry and rat TNF amplicons were joined together by SOEing PCR (PCR method) and gel purified using QIAquick (Qiagen) by following the manufacturer's instruction.

Meanwhile, pLVX-IB-EmGFP was digested with EcoRI-HF and BamHI-HF (NEB) to remove EmGFP. The linearized pLVX-IB vector was recombined with the mCherry-TNF using a cold fusion cloning kit (System Biosciences) by following the manufacturer's instructions. The resulting pLVX-IB-mCherry-TNF was used to transfect HEK293 FT cells to produce a recombinant virus and to generate RBL-2H3 cells (ATCC) that stably express mCherry-TNF, by following the same procedure described above.

### EXAMPLES

### Example 1

### Material and Methods

### Methods

Four vesicle/granule-anchored SNAREs (VAMP2, VAMP3, VAMP7, and VAMP8) and two Munc18 homologs (Munc18a and Munc18b) were systematically knocked down (KD) or knocked out in RBL-2H3 mast cells and antigen-induced release of 0-hexosaminidase, histamine, serotonin, and TNF was examined. Phenotypes were validated by rescue experiments. Immunofluorescence studies were performed to determine the subcellular distribution of key components.

### Results

The reduction of VAMP8 expression inhibited the exocytosis of b-hexosaminidase, histamine, and serotonin but not TNF. Unexpectedly, however, confocal microscopy revealed substantial co-localization between VAMP8 and TNF, and between TNF and serotonin. Meanwhile, the depletion of other VAMPs, including knockout of VAMP3, had no impact on the release of any of the mediators examined. On the other hand, TNF exocytosis was diminished specifically in stable Munc18b^{KD} cells, in a fashion that was rescued by exogenous, RNAi-resistant Munc18b. In line with this, TNF was co-localized with Munc18b (47%) to a much greater extent than with Munc18a (13%).

### Conclusion

Distinct exocytic pathways exist in mast cells for the release of different mediators.

### Antibodies

Anti-VAMP2 (104211), anti-VAMP7 (232011) mouse monoclonal antibodies and anti-VAMP8 (104303) rabbit polyclonal antibodies were purchased from Synaptic Systems. Anti-VAMP3 (Pab0055) and anti-Ykt6 (NBP2-94846) rabbit polyclonal antibodies were purchased from Covalab and Novus Biologicals respectively. Mouse anti-TNP IgE (557079) and anti-Munc18a mouse monoclonal IgG₁ (610336) and were purchased from BD Biosciences. Anti-Munc18b rabbit polyclonal antibody was custom made and affinity purified by GenScript. Anti-b-actin (SC-1616) goat polyclonal IgG, HRP-conjugated goat anti-mouse IgG (SC-2005), donkey anti-rabbit IgG (SC-2313), and donkey anti-goat IgG (SC-2033) were purchased from Santa Cruz Biotechnology. Monoclonal anti-serotonin antibody (5HT-H209) and goat anti-mouse IgG (Alexa Fluor™ 405; A5760) were purchased from Invitrogen.

### Cell Culture

Wild-type RBL-2H3 cells (ATCC) and derivatives were maintained in complete medium [DMEM medium containing 4.5 g/l D-Glucose and 110 mg/l sodium pyruvate (Gibco), 1× Glutamax (Gibco), and 10% heat inactivated FBS (Gibco)] at 37° C., 5% CO₂. RBL-2H3 Munc18a^{KD} and its control [26] were maintained in 5 μg/ml of puromycin, whereas RBL-2H3 Munc18b^{KD} and its control [26] were maintained in 700 μg/ml of G418.

### Generation of Stable Cell Lines

RNAi-resistant Munc18a or Munc18b cDNA carried by pLVX-EmGFP-IRES-Blasticidin [26] was introduced into RBL-2H3 Munc18a^{KD} or Munc18b^{KD} cells respectively via lentiviral transduction. To begin, HEK293-FT cells [26, 36] were grown in a 6-well plate overnight to about 70% confluency. After the removal of overnight medium, the HEK293-FT cells were incubated in 1 ml of complete medium plus 250 μl of Opti-MEM (Gibco)-based mixture that included 7.5 μl lipofectamine 3000 (Thermofisher), 10 μl of P3000 reagent (Thermofisher), 2.4 μg of psPAX2 lentiviral packaging plasmid [26, 36], 0.8 μg of pCMV-VSVG lentiviral envelope plasmid [36], and 1.8 μg of pLVX-Munc18a-EmGFP-IRES-Blasticidin, pLVX-Munc18b-EmGFP-IRES-Blasticidin, or pLVX-EmGFP-IRES-Blasticidin (for mock infection). Following 6 hours of incubation, cells continued to grow in 2 ml of fresh complete medium. After another 18 hours or 46 hours, the medium containing lentiviral particles were collected and centrifuged at 900 g for 5 min at 4° C. The supernatant was filtered through a 0.45-micron filter and stored at −80° C. To generate stable rescue cell lines, RBL-2H3 Munc18a^{KD} or Munc18b^{KD} cells grown to about 70% confluence in T-25 flasks were incubated with 7 ml of fresh complete medium [premixed with 8 μg/ml of Polybrene (Millipore)] and 1.2 ml of lentiviral particles (thawed from −80° C.). Following 24 hours of incubation at 37° C., 20 μg/ml of blasticidin was added to select cells for stably expressing RNAi-resistant Munc18 proteins.

To generate RBL-2H3 cells stably expressing mCherry-TNF rat TNF cDNA was amplified from TNF_Oral3562C_pMAL-c5E (GenScript) using 5′-TCCGGACTCAGA-TCTAGCACAGAAAGCATGAGCACGGAAAGCATG-3′ (SEQ ID NO: 2) and 5′-GGAGGGAGAGG-GGCGGGATCCTCACAGAGCAATGACTCC-3′ (SEQ ID NO: 3) as forward and reverse primers. mCherry was amplified from pCDH-TNF-SBP-mCherry (Addgene #65283; gift from Franck Perez) using using 5′-GGATCTATTTCCGGTGAATTCGCCACCATGGTGAGCAA-GGGCGAGG-3′(SEQ ID NO: 4) and 5′-CTGTGCTAGATCTGAGTCCGGACTTGTACAGCTCGTCC-ATGCCGC-3′ (SEQ ID NO: 5) as forward and reverse primers. Each PCR was conducted in a 50 μl mixture that contained 100 ng template DNA, 200 μM dNTPs, 1×pfu buffer, 5 units of Phusion DNA polymerase (G-Biosciences), and 200 nM of each primer. The mCherry and TNF amplicons were then joined together by SOEing PCR [37] and gel purified using QIAquick (Qiagen). Meanwhile, pLVX-EmGFP-IRES-Blasticidin was digested with EcoRI-HF (NEB) and BamHI-HF (NEB) to remove EmGFP. The rest of the vector was recombined with the mCherry-TNF using a cold fusion cloning kit (System Biosciences) by following the manufacturer's instructions. The resulting pLVX-mCherry-TNF-IRES-Blasticidin was used to transfect HEK293-FT cells to produce recombinant virus and to generate RBL-2H3 cells that stably expressed mCherry-TNF, by following the same procedure described in the previous paragraph.

To generate stable VAMP3 rescued RBL-2H3 VAMP3KO cells, rat VAMP3 was amplified from pMBP-TCS-VAMP3 [28] using PCR (forward primer: 5′-GGAATTCGCCACCATGTCTACAGGGGTG3′ (SEQ ID NO: 6), reverse primer: 5′-CGGGATCCAG-AGACACACCACACA-3′ (SEQ ID NO7)), and cloned into the EcoRI and BamHI sites of pLVX-EmGFP-TRES-Blasticidin. The resulting product, pLVX-VAMP3-EmGFP-IRES-Blasticidin, was verified by DNA sequencing and used to generate recombinant lentivirus to transduce RBL-2H3 VAMP3^{KO} cells by following the same procedure described above.

siRNA-Mediated Knockdown of VAMP Homologs

siGENOME siRNA oligos (SMARTpool format) for rat VAMP2 (M-090962-01-005), rat VAMP7 (M-094480-01-0005), or rat VAMP8 (M-099039-01-0005) were purchased from Dharmacon. VAMP3 siRNAs (s131634 and s131635) and Silencer Select Negative Control siRNA (4390843) were purchased from Ambion. siRNAs were introduced into RBL-2H3 cells via electroporation using SF Cell Line 4D-Nucleofector™ X Kit (V4XC-2032; Lonza). In brief, 1×10⁶ RBL-2H3 cells (counted in Invitrogen Countess II FL Automated Cell Counter) resuspended in 100 μl SF nucleofection solution were mixed gently with siRNA (1 μM for VAMP3, VAMP7, and VAMP8, or 2 μM for VAMP2) in a nucleofection cuvette. Electroporation was conducted using Program EQ151 in a Lonza 4D-Nucleofector™. Five hundred μl of prewarmed complete medium was added immediately to each cuvette and the mixtures were then transferred to a 24-well plate (containing 500 μl of complete medium) or a 6-well plate (containing 2 ml of complete medium). Cells were incubated at 37° C. for 24 hours for qPCR or 48 h for secretion assays.

To rescue phenotypes associated with VAMP8 KD, lentiviral vector pLV-EF1a-MCS-IRES-mCitrine or pLV-EF1a-VAMP8A-IRES-mCitrine [38] was utilized to produce lentiviral particles as described in the previous section. The lentiviral particles were added to RBL-2H3 cells that had been transfected with VAMP8 siRNA 3 h earlier (at 25 MOI). Secretion assays were performed following another 24 hours of incubation.

### CRISPR-Cas9 Mediated KO of VAMP3

Guide RNA (gRNA) targeting rat VAMP3 genome was designed using an online clustered regularly interspaced short palindromic repeats (CRISPR) gRNA design tool at IDT (https://www.idtdna.com). A 20-nucleotide sequence (5′-GAGTCTTCGATTACTGCCAG-3′) specifying a Cas9 cleavage site on exon 2 of the VAMP3 gene was selected based on the ranking of its on-target and off-target scores, and the corresponding oligo was cloned into the BbsI restriction site of sgRNA cloning vector pSpCas9(BB)-2A-GFP [aka PX458; Addgene #48138 (gift from Feng Zhang)].

In the meantime, the HR target vector (HR110PA-1) was purchased from System Biosciences to facilitate HDR (Homology Directed Repair)-based CRISPR knockout. A 600 bp homology arm matching either the left or the right side of the selected Cas9 cleavage site was amplified from RBL-2H3 genome via PCR. The primers for the left arm (forward primer: 5′-AAAACGACGGCCAGTGAATTCCTGGCTTGAGCAATCC-3′ (SEQ ID NO: 8); reverse primer: 5′-AAAACGACGGCCAGTGAATTCCTGGCTTGAGCAATCC-3′) and the primers for the right arm (forward primer: 5′-GAAATAACCTAGATCGGATCCGCACT-GGACCCTGAAG-3′ (SEQ ID NO: 9); reverse primer: 5′-GATTACGCCAAGCTTGCATGTGCTTCAG-ACTTTGGTC-3′ (SEQ ID NO: 10)) were designed to generate homologous arms 8 base pairs away from the Cas9 cleavage site. Using a cold fusion cloning kit (MC010B-1; System Biosciences), the left homologous arm was cloned into the MCS1 of the HR target vector (linearized by EcoRI and BglII double-digestion), after which the right homologous arm was cloned into the MCS2 of the HR target vector (linearized by BamHI and SphI double-digestion). The inserted homologous arms were verified by custom DNA sequencing (Europhins).

Next, PX458 plasmid containing VAMP3 gRNA (5 μg) and the HR target plasmid containing both homologous arms (5 μg) were mixed together to co-transfect RBL-2H3 cells via electroporation in Lonza 4D-Nucleofector™. Transfected cells were selected in complete medium containing 3 μg/ml of puromycin (added 48 h after electroporation). Using cloning discs (Scienceware), cells from each clone were transferred to a 24-well plate. The clones were then expanded in T-25 flasks and cells lysates were harvested for immunoblot analysis.

### Mast Cell Activation Via FcεRI

Sub-confluent RBL-2H3 cells (85%) grown on a 6-well plate were sensitized with 0.5 μg/ml anti-TNP IgE (BD Biosciences) and activated by 25 ng/ml TNP⁽²⁶⁾-BSA (Santa-Cruz) in RPMI 1640 (Corning) containing 0.1% BSA as described [21, 39]. The supernatant (around 1 ml) was collected from resting (no exposure to IgE) and activated (IgE and TNP-BSA) cells respectively and centrifuged at 500 g for 5 min at 4° C. to clear any cell debris. In the meantime, the cells attached to the bottom of the 6-well plates were lysed in 1 ml of cell culture medium, RPMI 1640 containing 0.1% BSA and 0.5% Triton X-100 for 5 min at RT. The lysate was centrifuged at 15,000 g for 5 min at 4° C. to clear any Triton-insoluble biomass. The cleared supernatant or lysate was separated into 3 aliquots: 800 μl were lyophilized for TNF assay; 50 μl were stored at −20° C. for serotonin and histamine assays; and the rest was used immediately for β-hexosaminidase assay.

### Secretion Assays

β-hexosaminidase activity and TNF protein level were measured as commonly used in the art [39]. To quantify histamine with an Enzyme Immunoassay (EIA) kit (EA213/96; Eagle Biosciences), the supernatant and cell lysates were diluted 100-fold in RPMI. Fifty μl of the diluted samples were then subject to acylation reaction and ELISA by following the manufacturer's instructions. To measure the level of serotonin, the supernatant and cell lysates were diluted 1:625 fold with standard buffer (EA630/96; Eagle Biosciences), 20 μl of which were subject to acylation and subsequent ELISA according to the manufacturer's instructions.

The absolute value for the exocytosis of each mediator (V) was expressed as the percentage of the signal released into the supernatant (M_{S}) relative to the total signal in the supernatant and in the lysate [V=M_{S}/(M_{S}+M_{L})%]. The value for IgE/allergen-dependent exocytosis was calculated by subtracting the value of the activated samples with the value of the resting samples (V_{E}=V_{A}−V_{R}). To minimize the impact of variation between biological repeats and to facilitate comparison of the levels of secretion between different mediators, V_{E} of the wild-type (or control) cells from each experiment was set to 100 and presented along with the relative values of KD, KO, or rescue cells.

### Quantitative Reverse Transcription PCR (RT-qPCR)

To estimate the efficacy of RNAi, siRNA-transfected cells in a 24-well plate (˜30% confluent) were briefly treated with 250 μl of 0.25% Trypsin-EDTA (Gibco) which was subsequently neutralized with 750 μl of complete medium. About 10⁵ cells were collected for RT-qPCR using TaqMan Gene Expression Cells-to-CT Kit (ThermoFisher Scientific). In brief, 22.5 μl of the cell lysate—prepared according to the manufacturer's instructions—were reverse transcribed in a 50 μl reaction mixture. Twenty μl of the reaction mixture were set aside for qPCR, exploiting CFX96 Real-Time PCR System (Bio-Rad). Fluorescently-labeled, target-specific probes (β-actin: Rn00667869_ml; VAMP2: Rn01465442_ml; VAMP3: Rn00588964_ml; VAMP7: Rn00585478_ml; VAMP8: Rn00582868_ml; and YKT6: Rn00581691_ml) were purchased from Thermofisher Scientific. The Bio-Rad CFX Maestro software was used to normalize the Cq values of VAMPs (against that of β-actin) and to determine the relative levels of mRNA expression.

### SDS-PAGE and Immunoblotting

RBL-2H3 cells grown to sub-confluency in a T-25 flask were rinsed twice with 8 ml of ice-cold phosphate-buffered saline (PBS) before they were resuspended in 500 μl of RIPA buffer [25 mM HEPES-NaOH (pH 7.4), 150 mM NaCl, 1% NP40 alternative, 1% deoxycholate, 0.1% SDS and 10 mM ethylenediaminetetra-acetic acid (EDTA)] that contained 1 mM phenylmethylsulfonyl fluoride (PMSF) and 1× Protease Inhibitor Cocktail (0.62 μg/ml leupeptin, 4 μg/ml pepstatin A, and 24.4 μg/ml pefabloc-SC). Following 1 min sonication (to shear chromosomal DNA), the protein concentration was measured in a Bradford assay. Cell lysates (50 μg of total protein) were then subject to 15% SDS-PAGE and immunoblot analysis. Primary antibodies were used at 1:1000 dilution unless otherwise specified. Horseradish peroxidase (HRP)-conjugated secondary antibodies were used at 1:5000 dilution. Immunoblots were developed in SuperSignal West Femto Maximum Sensitivity Substrate (Pierce).

### Immunofluorescence Staining and Confocal Microscopy

To assess the intracellular distribution of VAMP8, one million RBL-2H3 cells stably expressing mCherry-TNF were transfected with 5 μg of pEGFP-VAMP8 (Addgene #42311; gift from Thierry Galli) via electroporation as described above. For immunostaining, transfected or untransfected cells were grown to about 60% confluency in a 12-well plate (each well contained a sterile 18 mm diameter glass coverslip). Following medium removal, coverslips were washed three times with PBS and the cells on the coverslip were then fixed with 1 ml of 4% paraformaldehyde for 15 min. Following two additional washes with PBS, cells were permeabilized with 1 ml of 0.1% Triton X-100 in PBS for 15 min. Cells were then washed three times in PBS and five times in PBS containing 0.5% BSA. Afterwards, cells were blocked in 1 ml of PBS containing 2% BSA for 45 min. Cells were then washed five times with PBS containing 0.5% BSA, and incubated for 1 h in 250 μl of PBS containing 0.5% BSA and primary antibodies (concentrations specified in the figure legends). Following another five-time wash in PBS containing 0.5% BSA, cells were incubated for 1 h with 250 μl of PBS containing 0.5% BSA and 2.5 μg of goat anti-mouse IgG (Alexa Fluor™ 405). This was then followed by five washes in PBS containing 0.5% BSA. All incubations described above were conducted at RT and the volume for each wash was 1 ml. Coverslips were adhered to slides using Gelvatol mounting medium (https://cbi-pitt.webflow.io/protocols) and stored at 4° C. in dark until microscopic analysis. Confocal images were acquired using LASX software in Leica Stellaris Confocal Microscope equipped with 63×/1.4NA objective lens.

### Statistical Analysis

Secretion data were analyzed by paired Student t-test and shown as means±STD. For localization studies, the Pearson's correlation coefficient was calculated for each cell in 3D stack confocal microscopy images using the Coloc2 plugin in ImageJ. Data were analyzed by paired Student t-test and shown as means±SEM.

### Results

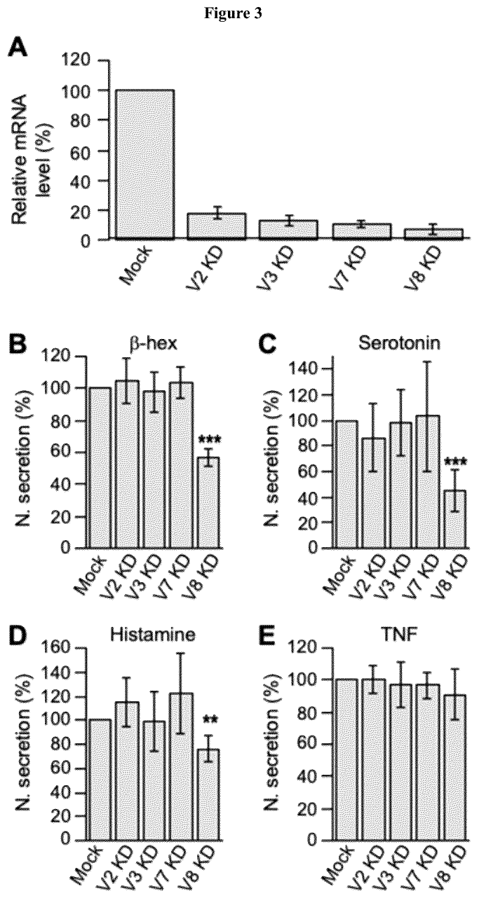

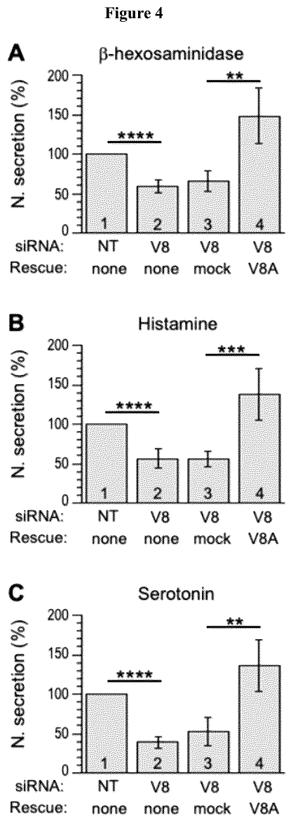

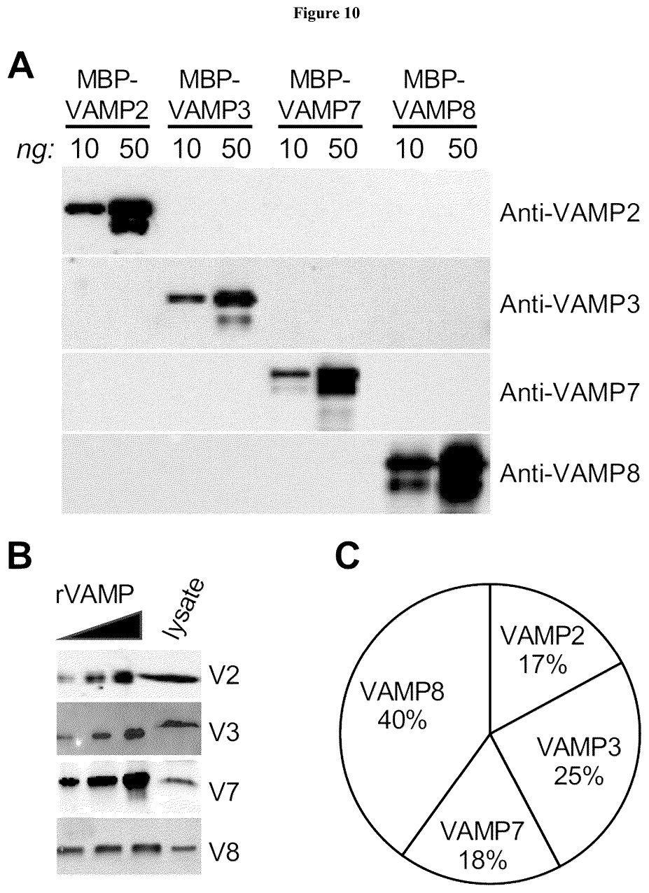

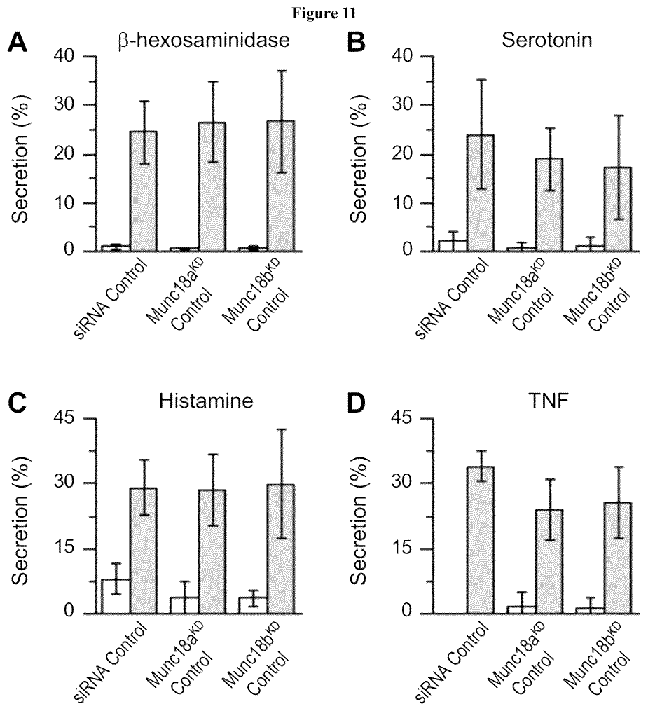

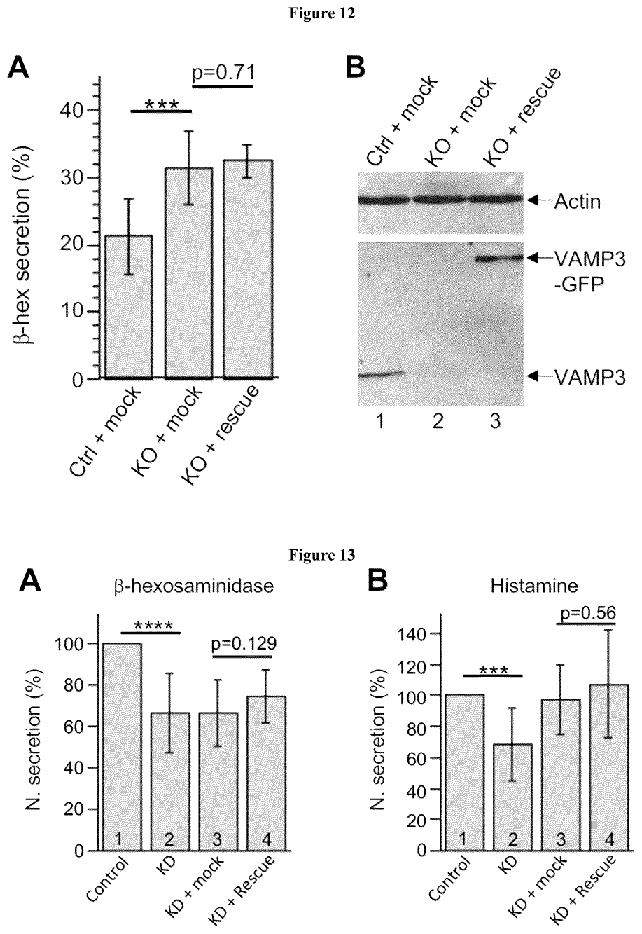

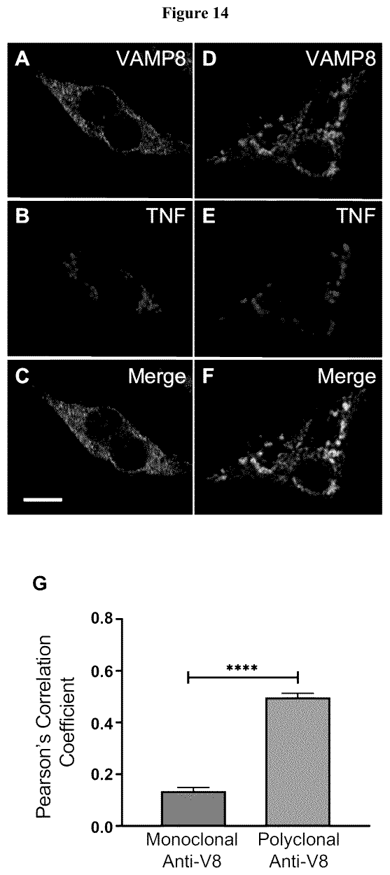

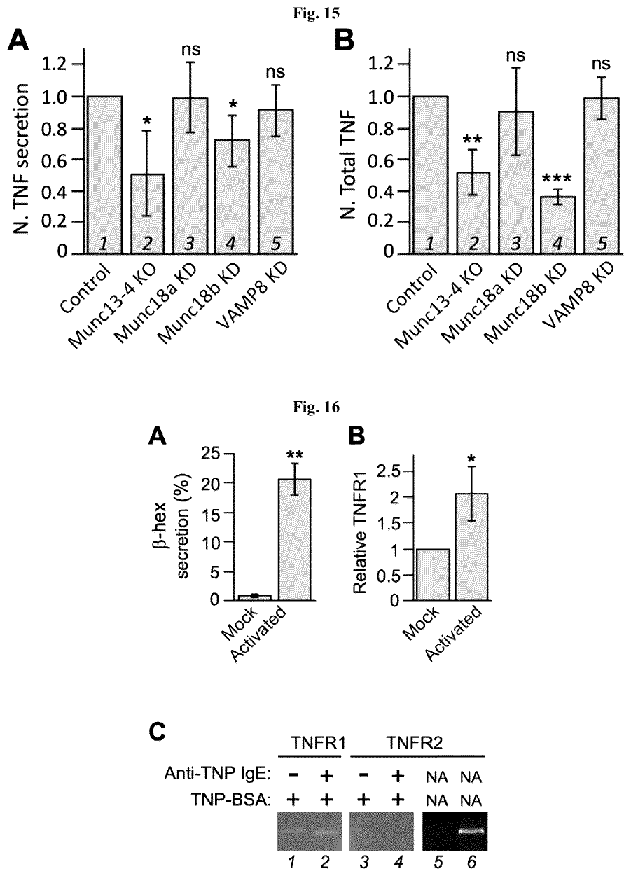

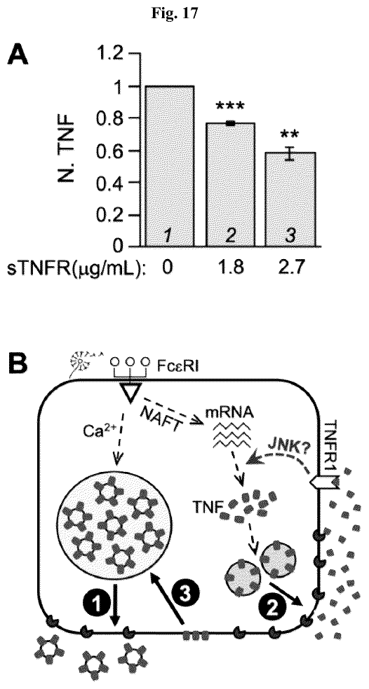

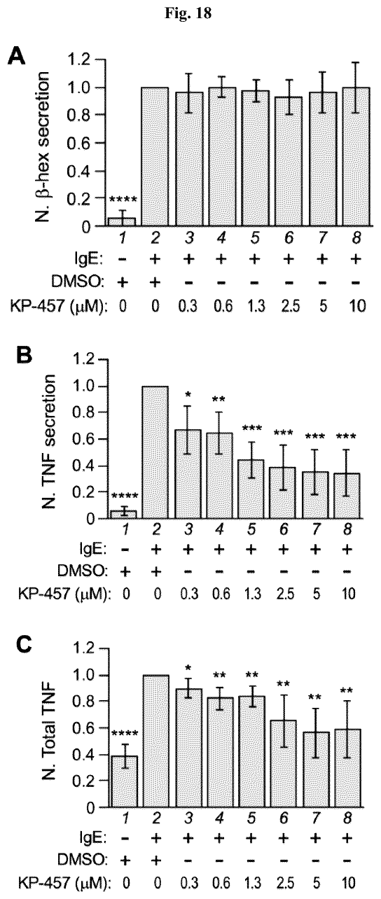

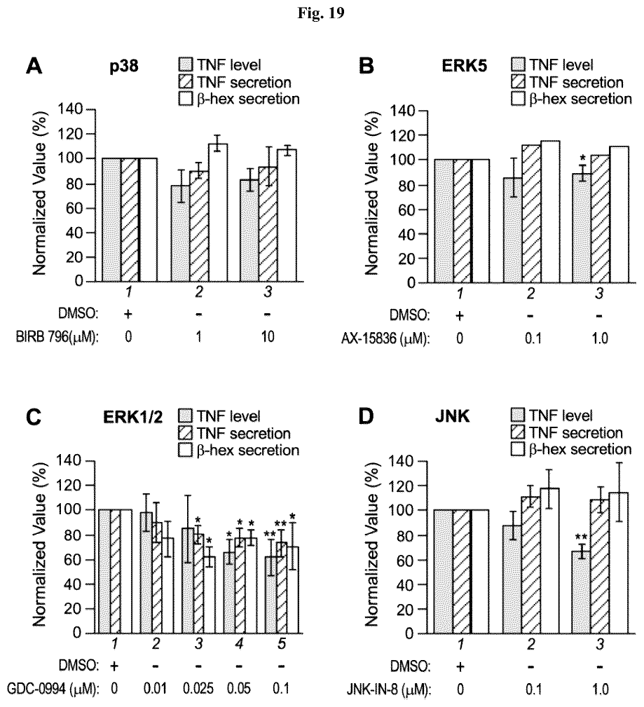

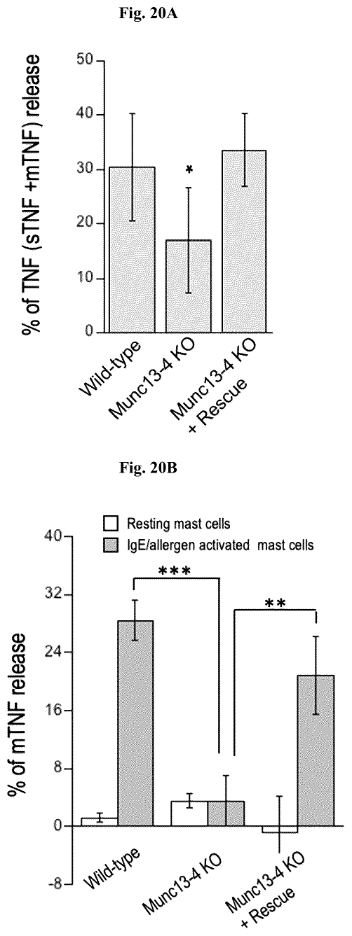

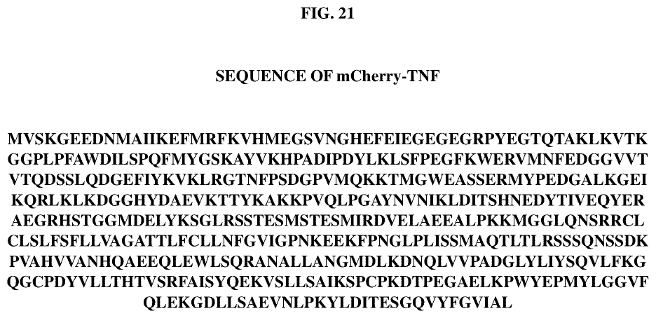

Selective requirement of VAMP8 in mediator release from allergen-triggered RBL-2H3 cells Mast cells express a number of v-SNAREs that are known to mediate exocytosis, including VAMP2, 3, 7, and 8. These four proteins are expressed in RBL-2H3 mast cells at a molar ratio of 17%:25%:18%:40% (FIG. 10), with VAMP8 being the most abundant. To examine their specific roles in allergen-induced release of mast cell mediators, we introduced siRNA into RBL-2H3 cells (via electroporation) to specifically target each of the four exocytic v-SNAREs. RT-qPCR indicated that the mRNA levels of VAMP2, 3, 7, and 8 were reduced by 80%, 85%, 90% and 95% respectively (FIG. 3A). Under our experimental conditions, wildtype RBL-2H3 cells transfected with non-target siRNA secreted approximately 24% of total β-hexosaminidase, 24% of total serotonin, 29% of total histamine, and 34% of total TNF after anti-TNP IgE/TNP-BSA challenge (FIG. 11). The partial silencing of VAMP2, 3 or 7 expressions, did not seem to reduce antigen-triggered mediator release (FIG. 3B to 3E). However, inhibition of VAMP8 transcription significantly diminished the exocytosis of β-hexosaminidase, histamine, and serotonin but not that of TNF (FIG. 3B to 3E). To assess if the secretion defects can be rescued, RNAi-resistant VAMP8Ala was introduced into siRNA-treated RBL-2H3 cells. Unlike the wild-type VAMP8, whose activity is suppressed by PKC-dependent phosphorylation, the phosphor-deficient VAMP8Ala is constitutively active when expressed in RBL-2H3 cells [38]. Under our experimental conditions, VAMP8Ala fully restored the secretion of 0-hexosaminidase, histamine, and serotonin (FIG. 4), demonstrating that the effects of VAMP8 siRNA were rather specific.

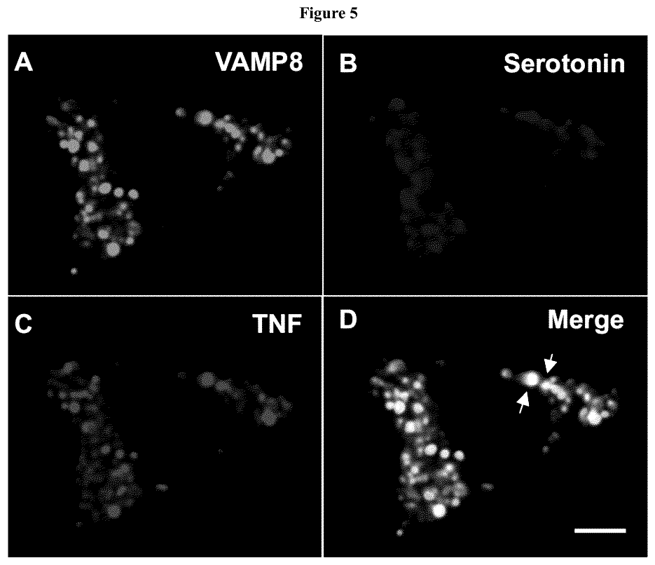

The secretion assays above suggest that in RBL-2H3 cells, TNF might be released from a secretory carrier that is devoid of VAMP8. To assess this, confocal microscopic studies were performed by exploiting an RBL-2H3 cell line that stably expresses mCherry-TNF (the endogenous TNF level in RBL-2H3 cells is too low for immunostaining). The distribution of classical secretory granules was monitored by a monoclonal antibody frequently used to mark serotonin in mast cells [16, 40]. The distribution of VAMP8 was determined based on the transient expression of EGFP-VAMP8. FIG. 5 shows substantial colocalization between VAMP8 and serotonin with the mean Pearson correlation coefficient at 0.45 (FIG. 5E). This is in agreement with an earlier observation that VAMP8 was required for serotonin release (FIG. 4C). There was also a noticeable overlap between TNF and serotonin signals in RBL-2H3 cells (FIG. 5), with the mean Pearson correlation coefficient at 0.52 (FIG. 5E), in agreement to the notion that at least a portion of TNF is prestored in the same secretory granules as serotonin [41]. The short of complete overlap between serotonin and TNF signals in RBL-2H3 cells could be an indication of different types of secretory granules in these cells. An unexpected finding from the confocal study is the rather extensive colocalization between VAMP8 and TNF, with the mean Pearson correlation coefficient at 0.58 (FIG. 5E). In addition, a considerable number of puncta positive for both VAMP8 and TNF were also highlighted by anti-serotonin (FIG. 5D, arrows).

Knocking Out VAMP3 does not Affect TNF Secretion from RBL-2H3 Cells

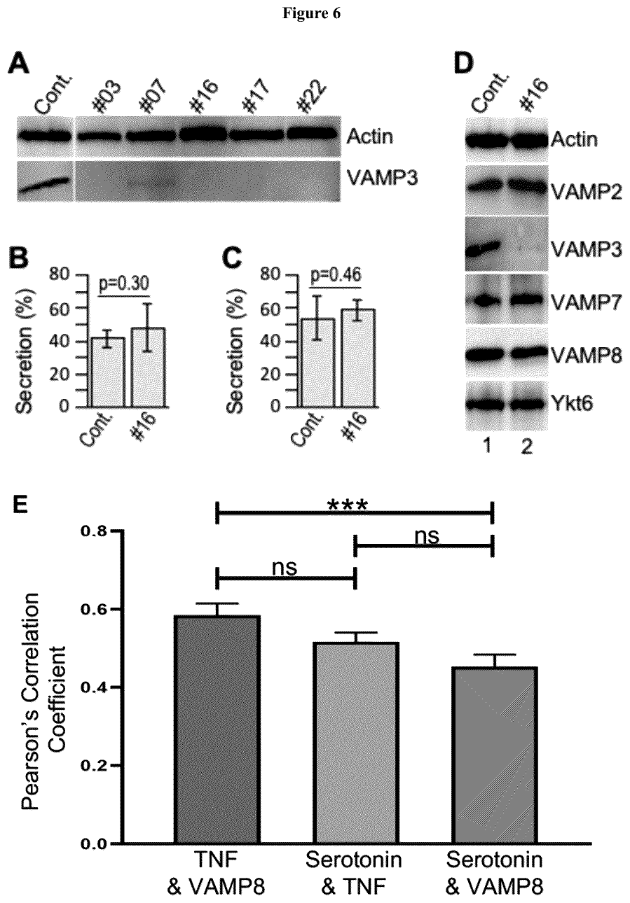

Because RNAi attenuated but did not eliminate the expression of VAMP2, 3, or 7, one cannot rule out their involvement in TNF release. Of the three, VAMP3 had been shown to mediate TNF secretion from both macrophages [42] and human synovial sarcoma cells [43]. To further test if VAMP3 might be responsible for TNF release from mast cells, an RBL-2H3 VAMP3^{KO} cell line was generated using CRISPR/Cas9-based technology. Out of 9 potential clones screened (via PCR), eight showed complete elimination of VAMP3 expression as determined by western blotting (FIG. 6A). Further testing was carried out to determine if knocking out VAMP3 causes TNF secretion defects in allergen-triggered RBL-2H3 cells. No reduction in TNF release was observed at either the 30 min or the 24 hour time point (FIGS. 6B & 6C). Instead, there is a statistically significant increase in β-hexosaminidase release at the 30 min time point (Supplementary FIG. 5A). However, this phenotype could not be rescued when an exogenous VAMP3-GFP was introduced (the C-terminally tagged GFP does not interfere with the function of VAMP3 [44]) into the VAMP3^{KO} cells (FIG. 12B).

To determine if the loss of VAMP3 was compensated by upregulation of other VAMP homologs, immunoblotting was performed to analyze the protein levels of other exocytic VAMPs in VAMP3^{KO} cells vs the control cells. No noticeable changes in the level of VAMP2, VAMP7, VAMP8, or Ykt6p were detected (FIG. 6D). These findings suggest that the secretory apparatus for TNF release is likely cell type.

### Differential Roles of Munc18a and Munc18b in Mast Cell Exocytosis

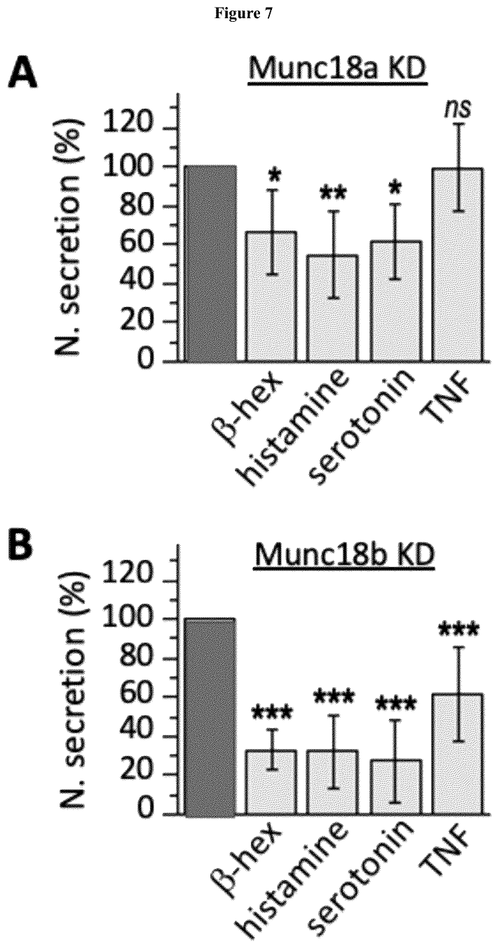

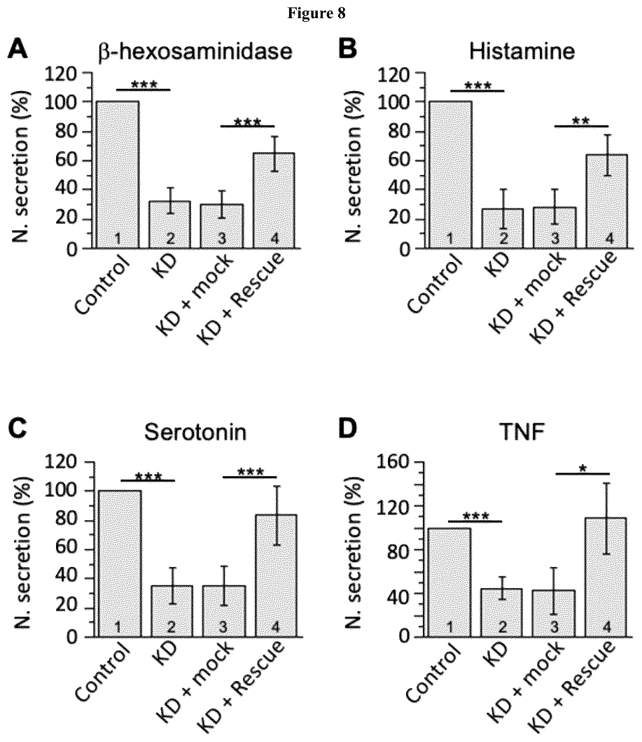

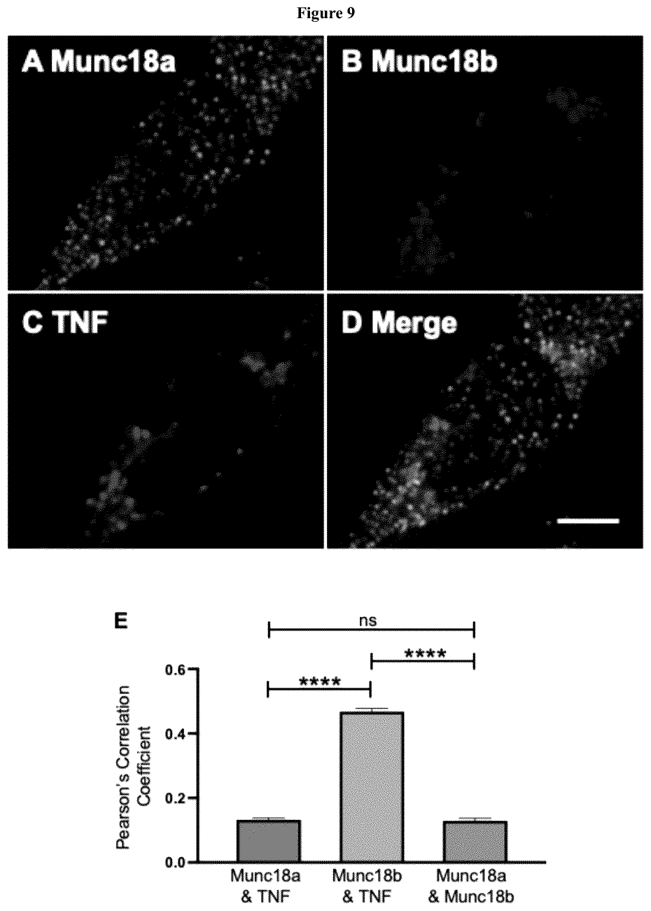

Since SM proteins work closely with SNAREs to regulate the specificity of membrane fusion [9, 13, 28], Munc18a and Munc18b were tested to see if they play differential roles in mediator release. In contrast to Munc18c, which does not appear to regulate mast cell exocytosis [45], both Munc18a and Munc18b are required for the maximal release of β-hexosaminidase from RBL-2H3 cells [26]. Using stable KD cells, it was observed that Munc18a depletion caused a modest but statistically significant reduction in allergen-triggered release of β-hexosaminidase, serotonin and histamine (FIG. 7A). However, TNF release was not affected in Munc18a^{KD} cells. Munc18b^{KD} cells, on the other hand, showed a more substantial reduction in the release of all four mediators (FIG. 7B). When RNAi-resistant Munc18b was introduced into the Munc18b^{KD} cells using lentiviral transduction, it showed that the secretion defects were either partially or fully rescued (FIG. 8). This unequivocally shows that Munc18b is responsible for the exocytosis of histamine, serotonin and TNF in IgE/allergen-activated mast cells. However, RNAi-resistant Munc18a was not able to restore the secretion in Munc18a^{KD} cells, for reasons that are still unclear (FIG. 13).

### Subcellular Localization of Munc18a and b in RBL-2H3 Cells

To determine the spatial distribution of Munc18 proteins, and to correlate that with the distribution of TNF, RBL-2H3 cells were immunostained stably expressing mCherry-TNF with antibodies specific to Munc18a or Munc18b. Munc18a and Munc18b are clearly distributed to distinct intracellular locations in resting cells (FIG. 9). Munc18b, which is required for TNF release (FIGS. 7 & 8), showed a substantial localization to mCherry-TNF positive compartments, with the mean Pearson correlation coefficient at 0.47 (FIG. 7E). In contrast, the colocalization between Munc18a and TNF was very low, with the mean Pearson correlation coefficient at 0.13 (FIG. 9E). These findings support the notion that Munc18b directly participates in the release of TNF from mast cells during allergic inflammation.

Munc13 Proteins Distinguish mTNF Release from sTNF Release

To determine if mTNF release and sTNF release require distinct regulators inside mast cells, Munc13-4 KO RBL-2H3 was used, which, according to ELISA, indicated that knocking out Munc13-4 partially inhibited IgE/allergen triggered TNF release (FIG. 20A). However, knocking out Munc13-4 completely eliminated mTNF release (FIG. 20B). Since there are three Munc13 homologs in RBL-2H3 mast cells, it is conceivable that a different Munc13 controls sTNF release whereas Munc13-4 controls mTNF release. Small molecule inhibitors of Munc13-4 (such as bexins) have been identified and could potentially be used to treat diseases linked to mTNF [Stephen Bruinsma et al 2018 JBC].

### Discussion

Despite the importance of mast cell exocytosis in health and disease, the identity of the underlying SNARE-SM machinery has been evasive. Using BMMC from VAMP8KO mice, one study suggested that VAMP8 was partially responsible for histamine release [17], whereas another study using the same approach showed the opposite [46]. A simple explanation is that the VAMP8^{KO} mice in the first study might have picked up an additional mutation that specifically inhibited histamine release. However, there is a remote possibility that the VAMP8^{KO} mice in the 2^{nd} study had picked up mutations that somehow enhanced histamine release. Regardless, the data present in FIGS. 3 & 4 support the conclusion that at least in RBL-2H3 mast cells, VAMP8 is required for the optimal release of both histamine and serotonin.

In addition to VAMP8, the present data demonstrated that knocking down Munc18b caused a significant reduction in the release of histamine (and other mediators), in a fashion that can be reversed by introducing exogenous, RNAi-resistant Munc18b. The data does not preclude Munc18a from catalyzing mast cell exocytosis in general or histamine release in particular. As a matter of fact, conditional knockout of the Munc18a gene from mast cells had a small but detectable impact on the rate of exocytic fusion based on time-resolved membrane capacitance measurements [47]. Moreover, knocking out Munc18b diminished but did not eliminate the amount of degranulated mast cells in mouse tissues during passive systemic anaphylaxis [47]. It is therefore plausible that Munc18a and Munc18b might promote different steps in the secretory pathway(s) [26].

While histamine, serotonin and β-hexosaminidase are mediators prestored in secretory granules in resting mast cells, TNF is both prestored and newly synthesized upon mast cell activation [5, 32-35]. Using the ELISA assay described above, an increased TNF level can be detected as early as 10 min after allergen-dependent activation, making it difficult to distinguish the release of TNF via the regulated secretory pathway (e.g., granule-based) versus the release of TNF via the constitutive secretory pathway (e.g., vesicle-based). Genetic studies of TNF release from primary mast cells (derived from KO mice) seem to have ruled out any involvement of syntaxin3 [45], syntaxin4 [45], or VAMP8 [17, 46], three SNAREs widely credited for the exocytosis of mast cell granules (aka degranulation) [48]. Using a functional analysis of VAMP8 exploiting RNAi has reinforced the view that VAMP8 is dispensable in TNF exocytosis in mast cells. Surprisingly, GFP-tagged VAMP8 was found to partially colocalize with serotonin and TNF (FIG. 5), which raised the question of why VAMP8 depletion reduced serotonin secretion but not that of TNF. To discern whether artifacts could have risen due to the overexpression of VAMP8, a major catalyst of endo/lysosomal trafficking [49-51], the present inventors have attempted to delineate the intracellular distribution of endogenous VAMP8 utilizing commercially available antibodies specific for the protein. As shown in FIG. 14, while immunostaining with a monoclonal antibody revealed little overlap between VAMP8 and TNF, immunostaining with a polyclonal antibody indicated substantial overlap between the two.

Since knocking out VAMP8 from bone marrow mononuclear cells (BMMCs) did not completely eliminate allergen-induced degranulation [17, 46], but instead increased VAMP3 expression [18], the involvement of VAMP3 in mediator release is conceivable. In fact, Mishima et al reported, in contrast to observations from the KO analysis (FIG. 6), that a stable VAMP3 KD RBL-2H3 cell line exhibited transient reduction in β-hexosaminidase release 30 min after allergen/IgE-mediated activation [52]. Given that this particular finding was not accompanied by rescue experiments, it would be premature to draw firm conclusions at this point. On the other hand, Mishima and colleagues did provide strong evidence that VAMP3 plays a role in both homotypic granule fusion and endocytosis of FcFRI (IgE receptor) [52]. Thus, depleting VAMP3 would have a dual effect on mast cell exocytosis; while it directly attenuates the rate of mast cell exocytosis/degranulation (by inhibiting homotypic granule fusion), it also enhances FcFRI-dependent signaling, and thereby indirectly upregulating mediator synthesis/release. This not only explains why the secretion defect in VAMP3KD cells was time-sensitive [52], it might also explain why no VAMP3-associated defects of mast cell exocytosis were observed in the KO studies.

### Fluorescence Assay Methodology

The present method employed the following methodology to identify mTNF and sTNF.

Protocol to Monitor mCherry-TNF Release from Mast Cells
1. Seed stable-expressing mcherry-TNF RBL-2H3 Cells in 12-well plate overnight in 1000 μl complete Dulbecco's Modified Eagle Medium (DMEM).
2. Remove media from cells grown to about 80% confluency.
3. Add 500 μl fresh media containing 0.5 μg/ml IgE-TNP or without (control).
4. Incubate for 3 h in 37 C with 5% CO₂.
5. Remove DMEM.
6. Wash cells with 1000 μl RPMI.
7. Wash cells with 500 μl 0.1% BSA in RPMI (RPMI/BSA).
8. Stimulate cells with 500 μl 25 ng/ml TNP-BSA in RPMI/BSA.
9. Incubate at 37 C with 5% CO2 for 30 minutes.
10. Withdraw supernatant and place on ice.
11. Centrifuge at 500 g for 5 minutes at 4° C.
12. Remove supernatant and place in new tubes on ice.
13. Re-suspend cells in 0.5% Triton X-100 in RPMI/BSA.
14. Incubate at RT for 4 minutes.
15. Resuspend and transfer to tubes on ice.
16. Centrifuge pellet at 15,000 g for 5 minutes at 4° C.
17. Remove supernatant and place in new tubes on ice.
18. Add 15 μl of supernatant and lysate in wells in black 384 well-plate with clear bottom (CORNING 3676).
19. Take fluorescence reading using Synergy HTX with monochromator channel using excitation=580, emission=610, and gain=150.
20. Subtract fluorescent intensity of samples from buffer controls.

It is possible to distinguish mTNF and sTNF using western blotting, however, this can be extremely time consuming and require many samples, as it is not very sensitive, as such, this methodology is not used to quantify TNF release. The above method employs ELISA to measure total TNF release in a first step, which includes both mTNF and sTNF. Next, the above secretion method is used to exclusively measure mTNF release, which is then subtracted from the total TNF value measured by ELISA.

### Example 2

### Materials and Methods

### 2.1. Cell Lines and RNAi

Wild-type RBL-2H3 cells were purchased from ATCC and maintained at 37° C. and 5% CO2 in Dulbecco's Modified Eagle Medium (DMEM) complete medium (Gibco) that contained 4.5 g/L D-Glucose, 110 mg/L sodium pyruvate, 1× Glutamax (Gibco), and 10% Fetal Bovine Serum (FBS) (Gibco; heat inactivated), without any antibiotics. Stable Munc18aKD and Munc18bKD cells were obtained from Sugita's lab and propagated according to the published protocol [83]. To transiently inhibit VAMP8 expression in RBL-2H3 cells, siGENOME siRNA oligos specific for VAMP8 (Dharmacon) were used to transfect sub-confluent RBL-2H3 cells as described [107].

### 2.2. Secretion Assays

RBL-2H3 cells and derivatives growing in 6-well plates were sensitized for 3 h with 0.5 μg/mL mouse anti-TNP (trinitrophenol phosphate) IgE (BD Pharmingen) and stimulated with 25 ng/mL of TNP(26)-BSA (Santa Cruz). β-hexosaminidase activities and TNF levels in the supernatant and the cell lysates were quantified as previously described [82]. Wherever indicated, sTNFR1 (Genscript) or KP-457 (Adooq Bioscience) was added along with TNP(26)-BSA during stimulation, and MAP kinase inhibitors (MedChemExpress) including Doramapimod/BIRB 796, AX-15836, Ravoxertinib/GDC-0994, and JNK-IN-8 were added 30 min ahead of TNP(26)-BSA addition. 2.3. Reverse transcription and PCRs RBL-2H3 cells (˜40% confluent) cultured in a 24-well plate were treated with 250 μL of 0.25% Trypsin-EDTA (Gibco) that was subsequently neutralized with 750 μL of complete medium. About 105 cells were collected for RT-qPCR (quantitative PCR) using TaqMan Gene Expression Cells-to-CT Kit (ThermoFisher Scientific) according to the manufacturer's instructions. Fluorescently labeled, target-specific probes ((3-actin: Rn00667869_ml; TNFR1: Rn01492348_ml; and TNFR2: Rn00709830_ml) were purchased from Thermofisher Scientific. The Cq values of TNFRs were standardized against that of β-actin utilizing the Bio-Rad CFX Maestro software and then the relative levels of mRNA expression were determined. For electrophoresis, TNFR1 was amplified from cDNA (prepared with the same TaqMan Gene Expression Cells-to-CT Kit described earlier) using AAAGAGGTGGAGGGTGAAG-3′ (SEQ ID NO: 11) and 5′-GCAGGTTCATGTCGCAAAG-3′ (SEQ ID NO: 12) as forward and reverse primers, whereas TNFR2 was amplified using 5′-ACACCCTACAAGCCAGAAC-3′ (SEQ ID NO: 13) and 5′-TCCTAACATCAGCAGACCC-3′ (SEQ ID NO: 14) as forward and reverse primers. PCR was conducted in a 20 μL mixture containing 1× One Taq Master Mix (NEB) and 200 nM of primers. Where specified, 1 μL of rat universal cDNA (BioChain) was used as positive control.

### Results and Discussion

It was previously demonstrated that the maximal production of TNF in antigen-activated RBL-2H3 cells requires Munc13-4 [82], a positive regulator of exocytic fusion. This, however, does not prove that exocytosis is connected to TNF production. Munc13-4 could have an unexpected moonlight function in the signaling pathways that promote TNF upregulation. To further investigate the link between exocytosis and TNF production, each of the three fusion catalysts known for mast cell exocytosis were knocked down, VAMP8 [84,85], Munc18a [83], and Munc18b [83]. Like Munc13-4, Munc18b depletion, which caused a reduction of allergen-triggered exocytosis of TNF and other mediators (e.g., β-hexosaminidase, histamine, and serotonin), also diminished the total level of TNF (FIG. 15, compare lanes 1 and 4). In contrast, RBL-2H3 cells depleted with either VAMP8 or Munc18a did not exhibit less TNF protein expression (FIG. 15B, compare lanes 3 and 5 to lane 1). Notably, these cells showed reduced mast cell exocytosis except for TNF (FIG. 15A, compare lanes 3 and 5 to lane 1). These findings argue that an autocrine loop exists in RBL-2H3 cells that couples TNF exocytosis to TNF production.

To determine which of the two TNFRs (there are only two mammalian TNFR genes) serves as the autocrine receptor, RT-qPCR was used to monitor the mRNA level of TNFR1 and TNFR2 in RBL-2H3 cells. TNFR1 expression was detected in both resting cells (virtually no detectable β-hexosaminidase secretion) and activated cells (20% β-hexosaminidase secretion), but its mRNA level in activated cells was twice as much as that in resting cells (FIGS. 16A and 16B). In comparison, Cook et al. [86] reported a 1-1.7 fold increase in TNFR1 expression in primary human conjunctival epithelial cells upon the addition of supernatants from allergen-activated conjunctival mast cells or pure IFNγ. TNFR2, on the other hand, was not detected at all in either resting cells or activated cells (FIG. 16C).

To assess the functional involvement of TNFR1 in TNF production, RBL-2H3 cells were incubated with sTNFR1 along with TNP-BSA after anti-TNP IgE-dependent sensitization. The purposes of sTNFR1 are to sequester any secreted TNF, preventing it from binding to the TNFR1 on the cell surface, and to interfere with the oligomerization/function of plasma membrane-anchored TNFR1 [87]. It was observed that sTNFR1 inhibited the overall level of TNF in a concentration dependent fashion (FIG. 17A), suggesting that the interaction between secreted TNF and cell surface TNFR1 is responsible for the augmentation of TNF protein levels in allergen-activated RBL-2H3 cells. Because according to the model shown in FIG. 17B, TNF upregulation in allergen-activated mast cells exploits both FcRI-mediated signaling and TNFR1-mediated signaling, the disruption of the autocrine loop alone (by sTNFR1) is expected to reduce but not eliminate TNF production, which is consistent with what was observed.

Although the full-length TNF (mTNF) prestored in multivesicular body (MVB) can be released as the membrane-bound form [88], newly synthesized TNF is delivered to the plasma membrane where it is released as the soluble form (sTNF) via TACE/ADAM17-dependent truncation [89,90]. To discern whether mTNF or sTNF serves as the autocrine agent, it was investigated if the inhibition of TNF truncation (and thus release) would affect TNF production by adding KP-457, a specific inhibitor for ADAM17 [91], in the secretion assay. KP-457 is expected to prevent ADAM17-mediated processing of mTNF at the cell surface, but it could also permeate cells to impede mTNF processing in intracellular compartments (if any) [92,93]. Either way, it would lead to increased mTNF at the cell surface but decreased sTNF in the supernatant. At concentrations between 0.3 M and 10 μM, KP-457 did not seem to affect the exocytosis of 3-hexosaminidase (FIG. 18A), but instead significantly reduced the amount of sTNF protein released into the supernatant (FIG. 18B). Furthermore, KP-457 inhibited the overall level of TNF in allergen-triggered RBL-2H3 cells (FIG. 18C). Together, these findings suggest that it is the specific release of TNF (not the other mast cell mediators) that led to the upregulation of TNF expression. This data also suggests that any mTNF accumulated on the cell membrane, as a result of ADAM17 inhibition, does not play a role in TNF autoregulation.

Although such feedback signaling is conceivable for any TNF producing cell that also expresses TNF receptors (e.g., monocytes and macrophages), the intracellular transduction underlying TNF auto regulation has never been experimentally elucidated or verified, except for the activation of transcription factor NF-κB [94-95]. However, the involvement of NF-κB in TNF expression is unlikely, despite the fact that NF-κB promotes the transcription of many cytokines. This is largely because the putative NF-κB binding sites in TNF promoter do not recognize NF-κB biochemically, or respond to it functionally [79,97]. Accordingly, it is believed that sTNF/TNFR1 interaction at mast cell surface promotes the post-transcriptional regulation of TNF expression via MAP kinase pathways (FIG. 17B), based on the following observations: i) many conventional MAP kinases are activated in TNF triggered cells [98,99]; ii) MAPK pathways have been shown to target various effector proteins to regulate TNF mRNA stability or translation [100-104]; and iii) inhibiting TNF/TNFR signaling in RBL-2H3 diminishes TNF protein level without reducing TNF mRNA level [82].

To assess the involvement of MAP kinases in TNF production in allergen-triggered RBL-2H3 cells, pharmaceuticals were exploited that selectively target each of the four conventional MAP kinase families including ERK1/2, p38, JNK, and ERK5 [105,106]. Under the experimental conditions, p38 inhibitor Doramapimod/BIRB 796 (targeting a, 3, 7, and 6 isoforms) did not seem to affect exocytosis or significantly reduce TNF proliferation (FIG. 19A), whereas ERK5 inhibitor AX-15836 caused a minor (10%) but statistically significant reduction in total TNF level (FIG. 19B). ERK1/2 inhibitor Ravoxertinib/GDC-0994, on the other hand, inhibited both mast cell exocytosis (judging by the secretion of 0-hexosaminidase and TNF) and the overall level of TNF in a seemingly correlated, dose-dependent manner (FIG. 19C). Intriguingly, JNK-IN-8 (targeting JNK1, 2, 3) diminished TNF level drastically without impacting TNF or β-hexosaminidase secretion (FIG. 19D), suggesting JNKs specifically regulate the signaling pathway(s) for TNF upregulation in RBL-2H3 cells.

### REFERENCES

All references cited herein are hereby incorporated by reference in their entirety as if fully set forth herein.

### Addin En.Reflist

1. Rao, K. N. and M. A. Brown, Mast cells: multifaceted immune cells with diverse roles in health and disease. Ann N Y Acad Sci, 2008. 1143: p. 83-104.
2. Gilfillan, A. M. and M. A. Beaven, Regulation of mast cell responses in health and disease. Crit Rev Immunol, 2011. 31(6): p. 475-529.
3. Wernersson, S. and G. Pejler, Mast cell secretory granules: armed for battle. Nat Rev Immunol, 2014. 14(7): p. 478-94.
4. Lundequist, A. and G. Pejler, Biological implications of preformed mast cell mediators. Cell Mol Life Sci, 2011. 68(6): p. 965-75.
5. Moon, T. C., A. D. Befus, and M. Kulka, Mast cell mediators: their differential release and the secretory pathways involved. Front Immunol, 2014. 5: p. 569.
6. Jahn, R. and R. H. Scheller, SNAREs—engines for membrane fusion. Nat Rev Mol Cell Biol, 2006. 7(9): p. 631-43.
7. Rothman, J. E., The principle of membrane fusion in the cell (Nobel lecture). Angew Chem Int Ed Engl, 2014. 53(47): p. 12676-94.
8. Han, J., K. Pluhackova, and R. A. Bockmann, The Multifaceted Role of SNARE Proteins in Membrane Fusion. Front Physiol, 2017. 8: p. 5.
9. Sudhof, T. C. and J. E. Rothman, Membrane fusion: grappling with SNARE and SM proteins. Science, 2009. 323(5913): p. 474-7.
10. Rizo, J. and T. C. Sudhof, The membrane fusion enigma: SNAREs, Sec1/Munc18proteins, and their accomplices—guilty as charged? Annu Rev Cell Dev Biol, 2012. 28: p. 279-308.
11. Xu, H., et al., HOPS prevents the disassembly of trans-SNARE complexes by Sec17p/Sec18p during membrane fusion. EMBO J, 2010. 29(12): p. 1948-60.
12. Baker, R. W., et al., A direct role for the Sec1 Munc18-family protein Vps33 as a template for SNARE assembly. Science, 2015. 349(6252): p. 1111-4.
13. Rathore, S. S., et al., Topological arrangement of the intracellular membrane fusion machinery. Mol Biol Cell, 2011. 22(14): p. 2612-9.
14. Jahn, R. and D. Fasshauer, Molecular machines governing exocytosis of synaptic vesicles. Nature, 2012. 490(7419): p. 201-7.
15. Woska, J. R., Jr. and M. E. Gillespie, Small-interfering RNA-mediated identification and regulation of the ternary SNARE complex mediating RBL-2H3 mast cell degranulation. Scand J Immunol, 2011. 73(1): p. 8-17.
16. Paumet, F., et al., Soluble NSF attachment protein receptors (SNAREs) in RBL-2H3 mast cells: functional role of syntaxin 4 in exocytosis and identification of a vesicle-associated membrane protein 8-containing secretory compartment. J Immunol, 2000. 164(11): p. 5850-7.
17. Tiwari, N., et al., VAMP-8 segregates mast cell-preformed mediator exocytosis from cytokine trafficking pathways. Blood, 2008. 111(7): p. 3665-74.
18. Tiwari, N., et al., Increased formation of VAMP-3-containing SNARE complexes in mast cells from VAMP-8 deficient cells. Inflamm Res, 2009. 58 Suppl 1: p. 13-4.
19. Brochetta, C., et al., Munc18-2 and syntaxin 3 control distinct essential steps in mast cell degranulation. J Immunol, 2014. 192(1): p. 41-51.
20. Sander, L. E., et al., Vesicle associated membrane protein (VAMP)-7 and VAMP-8, but not VAMP-2 or VAMP-3, are required for activation-induced degranulation of mature human mast cells. Eur J Immunol, 2008. 38(3): p. 855-63.
21. Suzuki, K. and I. M. Verma, Phosphorylation of SNAP-23 by IkappaB kinase 2 regulates mast cell degranulation. Cell, 2008. 134(3): p. 485-95.
22. Hibi, T., N. Hirashima, and M. Nakanishi, Rat basophilic leukemia cells express syntaxin-3 and VAMP-7 in granule membranes. Biochem Biophys Res Commun, 2000. 271(1): p. 36-41.
23. Guo, Z., C. Turner, and D. Castle, Relocation of the t-SNARE SNAP-23 from lamellipodia-like cell surface projections regulates compound exocytosis in mast cells. Cell, 1998. 94(4): p. 537-48.
24. Tadokoro, S., et al., Munc18-2 regulates exocytotic membrane fusion positively interacting with syntaxin-3 in RBL-2H3 cells. Mol Immunol, 2007. 44(13): p. 3427-33.
25. Martin-Verdeaux, S., et al., Evidence of a role for Munc18-2 and microtubules in mast cell granule exocytosis. J Cell Sci, 2003. 116(Pt 2): p. 325-34.
26. Bin, N. R., et al., Crucial role of the hydrophobic pocket region of Munc18 protein in mast cell degranulation. Proc Natl Acad Sci USA, 2013. 110(12): p. 4610-5.
27. Nigam, R., et al., Expression and transcriptional regulation of Munc18 isoforms in mast cells. Biochim Biophys Acta, 2005. 1728(1-2): p. 77-83.
28. Xu, H., M. G. Arnold, and S. V. Kumar, Differential Effects of Munc18s on Multiple Degranulation-Relevant Trans-SNARE Complexes. PLoS One, 2015. 10(9): p. e0138683.
29. Dvorak, A. M., Human mast cells. Adv Anat Embryol Cell Biol, 1989. 114: p. 1-107.
30. Raposo, G., et al., Accumulation of major histocompatibility complex class II molecules in mast cell secretory granules and their release upon degranulation. Mol Biol Cell, 1997. 8(12): p. 2631-45.
31. Theoharides, T. C., et al., Differential release of serotonin and histamine from mast cells. Nature, 1982. 297(5863): p. 229-31.
32. Gordon, J. R. and S. J. Galli, Mast cells as a source of both preformed and immunologically inducible TNF-alpha cachectin. Nature, 1990. 346(6281): p. 274-6.
33. Gordon, J. R. and S. J. Galli, Release of both preformed and newly synthesized tumor necrosis factor alpha (TNF-alpha) cachectin by mouse mast cells stimulated via the Fc epsilon RI. A mechanism for the sustained action of mast cell-derived TNF-alpha during IgE-dependent biological responses. J Exp Med, 1991. 174(1): p. 103-7.
34. Metcalfe, D. D., R. D. Peavy, and A. M. Gilfillan, Mechanisms of mast cell signaling in anaphylaxis. J Allergy Clin Immunol, 2009. 124(4): p. 639-46; quiz 647-8.
35. Rivera, J. and A. M. Gilfillan, Molecular regulation of mast cell activation. J Allergy Clin Immunol, 2006. 117(6): p. 1214-25; quiz 1226.
36. Bin, N. R., et al., C2 Domains of Munc13-4 Are Crucial for Ca(2+)-Dependent Degranulation and Cytotoxicity in NK Cells. J Immunol, 2018. 201(2): p. 700-713.
37. Horton, R. M., et al., Gene splicing by overlap extension. Methods Enzymol, 1993. 217: p. 270-9.
38. Malmersjo, S., et al., Phosphorylation of residues inside the SNARE complex suppresses secretory vesicle fusion. EMBO J, 2016. 35(16): p. 1810-21.
39. Ayo, T., et al., TNF Production in Activated RBL-2H3 Cells Requires Munc13-4. Inflammation, 2020. 43(2): p. 744-751.
40. Higashio, H., Y. I. Satoh, and T. Saino, Inhibitory role of Munc13-1 in antigen-induced mast cell degranulation. Biomed Res, 2017. 38(6): p. 321-329.
41. Blank, U., et al., Vesicular trafficking and signaling for cytokine and chemokine secretion in mast cells. Front Immunol, 2014. 5: p. 453.
42. Murray, R. Z., et al., A role for the phagosome in cytokine secretion. Science, 2005. 310(5753): p. 1492-5.
43. Boddul, S. V., et al., SNAP-23 and VAMP-3 contribute to the release of IL-6 and TNFalpha from a human synovial sarcoma cell line. FEBS J, 2014. 281(3): p. 750-65.
44. Caceres, P. S., et al., Vesicle-associated Membrane Protein 3 (VAMP3) Mediates Constitutive Trafficking of the Renal Co-transporter NKCC2 in Thick Ascending Limbs: ROLE IN RENAL FUNCTION AND BLOOD PRESSURE. J Biol Chem, 2016. 291(42): p. 22063-22073.
45. Sanchez, E., et al., Syntaxin 3, but not syntaxin 4, is required for mast cell-regulated exocytosis, where it plays a primary role mediating compound exocytosis. J Biol Chem, 2019. 294(9): p. 3012-3023.
46. Puri, N. and P. A. Roche, Mast cells possess distinct secretory granule subsets whose exocytosis is regulated by different SNARE isoforms. Proc Natl Acad Sci USA, 2008. 105(7): p. 2580-5.
47. Gutierrez, B. A., et al., Munc18-2, but not Munc18-1 or Munc18-3, controls compound and single-vesicle-regulated exocytosis in mast cells. J Biol Chem, 2018. 293(19): p. 7148-7159.
48. Lorentz, A., et al., The SNARE Machinery in Mast Cell Secretion. Front Immunol, 2012. 3: p. 143.
49. Wade, N., et al., Syntaxin 7 complexes with mouse Vps10p tail interactor Ib, syntaxin 6, vesicle-associated membrane protein (VAMP)8, and VAMP7 in b16 melanoma cells. J Biol Chem, 2001. 276(23): p. 19820-7.
50. Jean, S., et al., Starvation-induced MTMR13 and RAB21 activity regulates VAMP8 to promote autophagosome-lysosome fusion. EMBO Rep, 2015. 16(3): p. 297-311.
51. Pryor, P. R., et al., Combinatorial SNARE complexes with VAMP7 or VAMP8 define different late endocytic fusion events. EMBO Rep, 2004. 5(6): p. 590-5.
52. Mishima, S., et al., Multifunctional regulation of VAMP3 in exocytic and endocytic pathways of RBL-2H3 cells. Front Immunol, 2022. 13: p. 885868.
53. S. C. Bischoff, et al., Mast cells are an important cellular source of tumour necrosis factor alpha in human intestinal tissue, Gut 44 (5) (1999) 643-652.
54. R. E. Dery, et al., Redundancy or cell-type-specific regulation? Tumour necrosis factor in alveolar macrophages and mast cells, Immunology 99 (3) (2000) 427-434.
55. D. Izadi, et al., Identification of TNFR2 and IL-33 as therapeutic targets in localized fibrosis, Sci. Adv. 5 (12) (2019) eaay0370.
56. R. Schulz, G. Heusch, Tumor necrosis factor-alpha and its receptors 1 and 2: Yin and Yang in myocardial infarction? Circulation 119 (10) (2009) 1355-1357.
57. R. Aoki, et al., Mast cells play a key role in host defense against herpes simplex virus infection through TNF-alpha and IL-6 production, J. Invest. Dermatol. 133 (9) (2013) 2170-2179.
58. B. Echtenacher, D. N. Mannel, L. Hultner, Critical protective role of mast cells in a model of acute septic peritonitis, Nature 381 (6577) (1996) 75-77.
59. C. Liu, et al., Tumor necrosis factor-alpha is required for mast cell-mediated host immunity against cutaneous Staphylococcus aureus infection, J. Infect. Dis. 218 (1) (2018) 64-74.
60. R. Malaviya, et al., Mast cell modulation of neutrophil influx and bacterial clearance at sites of infection through TNF-alpha, Nature 381 (6577) (1996) 77-80.
61. S. Nakae, et al., Mast cells enhance T cell activation: importance of mast cell costimulatory molecules and secreted TNF, J. Immunol. 176 (4) (2006) 2238-2248.
62. S. Nakae, et al., Mast cells enhance T cell activation: importance of mast cell-derived TNF, Proc. Natl. Acad. Sci. U.S.A 102 (18) (2005) 6467-6472.
63. K. N. Atretkhany, et al., Distinct modes of TNF signaling through its two receptors in health and disease, J. Leukoc. Biol. 107 (6) (2020) 893-905.
64. S. Reuter, et al., Mast cell-derived tumour necrosis factor is essential for allergic airway disease, Eur. Respir. J. 31 (4) (2008) 773-782.
65. C. Brightling, M. Berry, Y. Amrani, Targeting TNF-alpha: a novel therapeutic approach for asthma, J. Allergy Clin. Immunol. 121 (1) (2008) 5-10, quiz 11-2.
66. C. Russo, R. Polosa, TNF-alpha as a promising therapeutic target in chronic asthma: a lesson from rheumatoid arthritis, Clin. Sci. (Lond.) 109 (2) (2005) 135-142.
67. B. Beutler, Regulation of cachectin biosynthesis occurs at multiple levels, Prog. Clin.
   Biol. Res. 349 (1990) 229-240.
68. J. Han, P. Thompson, B. Beutler, Dexamethasone and pentoxifylline inhibit endotoxin-induced cachectin/tumor necrosis factor synthesis at separate points in the signaling pathway, J. Exp. Med. 172 (1) (1990) 391-394.
69. M. B. Olszewski, et al., Efficient sorting of TNF-alpha to rodent mast cell granules is dependent on N-linked glycosylation, Eur. J. Immunol. 36 (4) (2006) 997-1008.
70. K. Mukai, et al., Mast cells as sources of cytokines, chemokines, and growth factors, Immunol. Rev. 282 (1) (2018) 121-150.
71. P. Draber, et al., Signal transduction and chemotaxis in mast cells, Eur. J. Pharmacol. 778 (2016) 11-23.
72. I. Ohno, et al., Gene expression and production of tumour necrosis factor by a rat basophilic leukaemia cell line (RBL-2H3) with IgE receptor triggering, Immunology 70 (1) (1990) 88-93.
73. Y. Gon, S. Nunomura, C. Ra, Common and distinct signalling cascades in the production of tumour necrosis factor-alpha and interleukin-13 induced by lipopolysaccharide in RBL-2H3 cells, Clin. Exp. Allergy 35 (5) (2005) 635-642.
74. T. Hochdorfer, et al., LPS-induced production of TNF-alpha and IL-6 in mast cells is dependent on p38 but independent of TTP, Cell. Signal. 25 (6) (2013) 1339-1347.
75. V. Supajatura, et al., Differential responses of mast cell Toll-like receptors 2 and 4 in allergy and innate immunity, J. Clin. Invest. 109 (10) (2002) 1351-1359.
76. J. D. McCurdy, et al., Cutting edge: distinct Toll-like receptor 2 activators selectively induce different classes of mediator production from human mast cells, J. Immunol. 170 (4) (2003) 1625-1629.
77. S. Varadaradjalou, et al., Toll-like receptor 2 (TLR2) and TLR4 differentially activate human mast cells, Eur. J. Immunol. 33 (4) (2003) 899-906.
78. Y. Ohkawara, et al., Human lung mast cells and pulmonary macrophages produce tumor necrosis factor-alpha in sensitized lung tissue after IgE receptor triggering, Am. J. Respir. Cell Mol. Biol. 7 (4) (1992) 385-392.
79. J. V. Falvo, A. V. Tsytsykova, A. E. Goldfeld, Transcriptional control of the TNF gene, Curr. Dir. Autoimmun. 11 (2010) 27-60.
80. M. Klein, et al., Specific and redundant roles for NFAT transcription factors in the expression of mast cell-derived cytokines, J. Immunol. 177 (10) (2006) 6667-6674.
81. A. Lorentz, et al., The SNARE machinery in mast cell secretion, Front. Immunol. 3 (2012) 143.
82. T. E. Ayo, et al., TNF production in activated RBL-2H3 cells requires Munc13-4, Inflammation 43 (2) (2020) 744-751.
83. N. R. Bin, et al., Crucial role of the hydrophobic pocket region of Munc18 protein in mast cell degranulation, Proc. Natl. Acad. Sci. U.S.A 110 (12) (2013) 4610-4615.
84. N. Tiwari, et al., VAMP-8 segregates mast cell-preformed mediator exocytosis from cytokine trafficking pathways, Blood 111 (7) (2008) 3665-3674.
85. N. Puri, P. A. Roche, Mast cells possess distinct secretory granule subsets whose exocytosis is regulated by different SNARE isoforms, Proc. Natl. Acad. Sci. U.S.A 105 (7) (2008) 2580-2585.
86. E. B. Cook, et al., Regulation of the receptor for TNFalpha, TNFR1, in human conjunctival epithelial cells, Invest. Ophthalmol. Vis. Sci. 49 (9) (2008) 3992-3998.
87. C. Karathanasis, et al., Single-molecule imaging reveals the oligomeric state of functional TNFalpha-induced plasma membrane TNFR1 clusters in cells, Sci. Signal. 13 (614) (2020).
88. H. G. Zhang, et al., A membrane form of TNF-alpha presented by exosomes delays T cell activation-induced cell death, J. Immunol. 176 (12) (2006) 7385-7393.
89. A. Sommer, et al., Extracellular sphingomyelinase activity impairs TNF-alpha-induced endothelial cell death via ADAM17 activation and TNF receptor 1 shedding, Oncotarget 8 (42)(2017)72584-72596.
90. M. J. Mohan, et al., The tumor necrosis factor-alpha converting enzyme (TACE): a unique metalloproteinase with highly defined substrate selectivity, Biochemistry 41 (30) (2002) 9462-9469.
91. S. Hirata, et al., Selective inhibition of ADAM17 efficiently mediates glycoprotein Ibalpha retention during ex vivo generation of human induced pluripotent stem cell-derived platelets, Stem Cells Transl. Med. 6 (3) (2017) 720-730.
92. R. A. Black, et al., A metalloproteinase disintegrin that releases tumour-necrosis factor-alpha from cells, Nature 385 (6618) (1997) 729-733.
93. H. J. Robertshaw, F. M. Brennan, Release of tumour necrosis factor alpha (TNFalpha) by TNFalpha cleaving enzyme (TACE) in response to septic stimuli in vitro, Br. J. Anaesth. 94 (2) (2005) 222-228.
94. N. Verma, et al., Silencing of TNF-alpha receptors coordinately suppresses TNF-alpha expression through NF-kappaB activation blockade in THP-1 macrophage, FEBS Lett. 583 (17) (2009) 2968-2974.
95. J. Xaus, et al., LPS induces apoptosis in macrophages mostly through the autocrine production of TNF-alpha, Blood 95 (12) (2000) 3823-3831.
96. J. M. Gane, R. A. Stockley, E. Sapey, TNF-alpha autocrine feedback loops in human monocytes: the pro- and anti-inflammatory roles of the TNF-alpha receptors support the concept of selective TNFR1 blockade in vivo, J Immunol Res 2016 (2016) 1079851.
97. T. Ishizuka, et al., Mast cell tumor necrosis factor alpha production is regulated by MEK kinases, Proc. Natl. Acad. Sci. U.S.A 94 (12) (1997) 6358-6363.
98. G. Sabio, R. J. Davis, TNF and MAP kinase signalling pathways, Semin. Immunol. 26 (3) (2014) 237-245.
99. K. Loupasakis, et al., Tumor necrosis factor dynamically regulates the mRNA stabilome in rheumatoid arthritis fibroblast-like synoviocytes, PLoS One 12 (7) (2017), e0179762.
100. M. V. Pashenkov, et al., The role of the p38-MNK-eIF4E signaling Axis in TNF production downstream of the NOD1 receptor, J. Immunol. 198 (4) (2017) 1638-1648.
101. M. Buxade, et al., The Mnks are novel components in the control of TNF alpha biosynthesis and phosphorylate and regulate hnRNP A1, Immunity 23 (2) (2005) 177-189.
102. E. Carballo, W. S. Lai, P. J. Blackshear, Feedback inhibition of macrophage tumor necrosis factor-alpha production by tristetraprolin, Science 281 (5379) (1998) 1001-1005.
103. B. Gonzalez-Teran, et al., Eukaryotic elongation factor 2 controls TNF-alpha translation in LPS-induced hepatitis, J. Clin. Invest. 123 (1) (2013) 164-178.
104. P. Gais, et al., TRIF signaling stimulates translation of TNF-alpha mRNA via prolonged activation of MK2, J. Immunol. 184 (10) (2010) 5842-5848.
105. M. Cargnello, P. P. Roux, Activation and function of the MAPKs and their substrates, the MAPK-activated protein kinases, Microbiol. Mol. Biol. Rev. 75 (1) (2011) 50-83.
106. D. K. Morrison, MAP kinase pathways, Cold Spring Harb. Perspect. Biol. 4 (11) (2012).
107. P. Adhikari, et al., Exocytic machineries differentially control mediator release from allergen-triggered RBL-2H3 cells, Inflammation Res (2023), https://doi.org/10.1007/s00011-023-01698-z.

What is claimed is:

1. A method for inhibiting the release of tumor necrosis factor (TNF) from mast cells, comprising steps of.
a) administering an effective amount of an inhibitor to a subject; wherein the inhibitor is an inhibitor that selectively targets conventional mitogen activated protein kinase signaling pathways; and
b) allowing the inhibitor to interact with mast cells or their signaling pathways involved in TNF release, thereby inhibiting TNF release.

2. The method of claim 1, wherein the mitogen activated protein kinase inhibitor is selected from the group consisting of MEK inhibitors, BRAF inhibitors, Dual BRAF and MEK inhibitors, ERK Inhibitors, INK Inhibitors, p38 MAPK Inhibitors, and TNF-alpha converting enzyme (TACE) inhibitor.

3. The method of claim 1, wherein the inhibitor is selected from the group consisting of JNK inhibitors.

4. A method for preventing the conversion of membrane-bound tumor necrosis factor (mTNF) to soluble tumor necrosis factor (sTNF) in mast cells using an inhibitor, comprising steps of.
a) administering an effective amount of the inhibitor to a subject; wherein the inhibitor is a chemical compound that selectively targets conventional mitogen activated protein kinase signaling pathways; and
b) allowing the inhibitor to interact with mast cells or the enzymatic machinery responsible for the cleavage of mTNF within mast cells, thereby inhibiting the conversion of mTNF to sTNF.

5. The method of claim 4, wherein the mitogen activated protein kinase inhibitor is selected from the group consisting of MEK inhibitors, BRAF inhibitors, Dual BRAF and MEK inhibitors, ERK Inhibitors, INK Inhibitors, p38 MAPK Inhibitors, and TNF-alpha converting enzyme (TACE) inhibitor.

6. The method of claim 4, wherein the inhibitor is selected from the group consisting of JNK inhibitors.

7. A method for treating a disease or disorder caused by mast cell tumor necrosis factor, comprising a step of.
administering an effective amount of a pharmaceutical composition to a subject wherein the pharmaceutical composition comprises an inhibitor that selectively targets conventional mitogen activated protein kinase signaling pathways.

8. The method of claim 7, wherein the disease is selected from the group consisting of asthma, allergic rhinitis, atopic dermatitis, inflammatory bowel disease, rheumatoid arthritis, psoriasis, anaphylaxis, systemic mastocytosis, chronic urticaria, and autoimmune disorders.

9. The method of claim 7, wherein the mitogen activated protein kinase inhibitor is selected from the group consisting of MEK inhibitors, BRAF inhibitors, Dual BRAF and MEK inhibitors, ERK Inhibitors, INK Inhibitors, p38 MAPK Inhibitors, and TNF-alpha converting enzyme (TACE) inhibitor.

10. The method of claim 7, wherein the inhibitor is selected from the group consisting of TNF-alpha converting enzyme inhibitor KP-457; ERK1/2 extracellular signal-regulated kinase 1/2 Ravoxertinib (GDC-0994); and small molecule inhibitor JNK-IN8.

11. The method of claim 7, wherein the inhibitor is selected from the group consisting of JNK inhibitors.
